(19) United States
(12) Patent Application Publication
(10) Pub. No.: US 2020/0079729 A1
(43) Pub. Date: Mar. 12, 2020

(54) PHENYL UREA ANALOGS AS FORMYL PEPTIDE RECEPTOR 1 (FPR1) SELECTIVE AGONISTS

(71) Applicant: Allergan, Inc., Irvine, CA (US)

(72) Inventors: Tien T. Duong, Rancho Santa Margarita, CA (US); Richard L. Beard, Newport Beach, CA (US); Michael E. Garst, Newport Beach, CA (US)

(21) Appl. No.: 15/746,918

(22) PCT Filed: Aug. 2, 2016
(86) PCT No.: PCT/US2016/045114

§ 371 Date: Jan. 23, 2018

Related U.S. Application Data
(60) Provisional application No. 62/201,387, filed on Aug. 5, 2015.

(51) Int. Cl. C07C 275/30; C07K 5/062

(52) U.S. Cl. CPC C07C 275/30; C07K 5/06026

(57) ABSTRACT

The present disclosure relates to phenyl urea derivatives, processes for preparing them, pharmaceutical compositions containing them and their use as pharmaceuticals as modulators of the N-formyl peptide receptor (FPR), such as agonism of the FPR1 and/or FPR2 receptor, or selective agonism of the FPR1 receptor relative to the FPR2 receptor.

### FIELD OF THE INVENTION

The present invention relates to phenyl urea derivatives, processes for preparing them, pharmaceutical compositions containing them and their use as pharmaceuticals as modulators of N-formyl peptide receptor(s) (FPR(s)), such as modulators of the N-formyl peptide receptor 1 (FPR1) and/or the N-formyl peptide receptor 2 (FPR2; also known as FPRL-1 or ALXA4), or as selective modulators of FPR1 relative to FPR2. The invention relates specifically to the use of these compounds and their pharmaceutical compositions to treat disorders associated with FPR modulation, such as FPR1 and/or FPR2 agonism, or selective agonism of FPR1 relative to FPR2.

### BACKGROUND OF THE INVENTION

The FPR family belongs to the seven transmembrane domain G-protein-coupled receptor (GPCR) family. There are three members of this family in humans, including FPR1 and FPR2. FPRs are critical regulators of host defense in phagocytosis, and are considered highly relevant factors for the chemotaxis of immune cells. In view of their ability to promote the resolution of inflammation, these receptors represent an important “pro-resolutionary” molecular target for the development of new therapeutic agents in diseases or conditions involving excessive inflammatory responses.

WO 2014/138037 A1 discloses methods of treating ocular inflammatory diseases by administering a pharmaceutical composition comprising an FPR agonist; WO 2014/138046 A1 discloses methods of treating dermal inflammation and dermal diseases by administering a pharmaceutical composition comprising an FPR agonist; and US 2013/0109866 discloses compounds of the general structure below (with the variable “R” groups as defined therein) as FPR modulators for the treatment of a variety of diseases or conditions, including ocular and dermal inflammatory diseases and conditions:

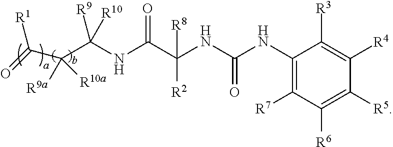

The entire disclosure of each of the preceding references is incorporated herein by this specific reference.

FPR2 is expressed predominantly on inflammatory cells, such as monocytes and neutrophils, as well as on T cells, and has been shown to play a critical role in leukocyte trafficking during inflammation and human pathology (see Chiang N, Serhan C N, Dahlen, S, Drazen J M, Hay D W P, Rovati E, Shimizu T, Yokomizo T, Brink, C. The lipoxin receptor ALX: Potent ligand-specific and stereoselective actions in vivo. Pharmacological Reviews 2006; 58: 463-519).

FPRs are also expressed by immune cells of the central nervous system (CNS), and FPR expression is up-regulated during bacterial meningitis. Lack of FPR1 and FPR2 leads to more severe inflammation and higher mortality in mice infected with Streptococcus pneumonia within the CNS, suggesting that these FPRs play an important role in the innate response against this pathogen in the CNS (Oldekamp, S. et al., Immunology, 143(3), pp. 447-461, 2014).

FPR1 and FPR2 mediate rapid neutrophil mobilization to accelerate wound healing, as shown in Listeria-infected mice. These FPRs sense pathogen-derived chemotactic ligands and recognize host-derived chemotactic peptides in inflammation and injury. The FPRs promote the healing of sterile skin wounds in mice by initiating neutrophil infiltration (Liu, M. et al., PLoS One, 9(6): e90613, 2014). FPRs have also been shown to guide the first wave of neutrophil infiltration in livers of Listeria-infected mice to effectively eliminate the invading pathogen (Liu, M. et al., Sci. Rep., Vol 2, pp. 786, 2012). The FPRs appear to play a prominent role in regulating the hepatic inflammatory response after LPS induced liver injury; for example, FPR1 and FPR2 deficiency has been associated with increased inflammation and enhanced liver injury after LPS stimulation (Giebeler, A. et al., PLoS One, 9(6): e100522, 2014).

During intestinal mucosal injury, a complex array of proinflammatory and protective mechanisms regulates inflammation and severity. Controlling inflammatory responses and promoting epithelial restitution and barrier recovery requires secretion of anti-inflammatory mediators (Babbin, B. A. et al., J. Immunol., 208, 181(7), pp. 5035-5044). FPR1, a chemo-attractant receptor expressed mainly on leukocytes, is expressed in epithelia, and an FPR1/NADPH oxidase (NOX1)-dependent redox signaling pathway that promotes mucosal wound repair has been delineated in intestinal epithelia. Specific gut microbiota stimulate FPR1 on intestinal epithelial cells, generating reactive oxygen species via enterocyte NOX1, causing rapid phosphorylation of focal adhesion kinase (FAK) and extracellular signal-regulated kinase mitogen-activated protein kinase, which together stimulate migration and proliferation of enterocytes adjacent to colonic wounds. FPR1 was thus identified as a pattern recognition receptor for perceiving the enteric microbiota that promote mucosal wound repair by generating reactive oxygen species from the enterocyte NOX1. (See Leoni, G. et al., J. Clin. Invest., Vol 123, pp. 443-454, 2013; Alam, A. et al., Mucosal Immunol., 2014, 7(3), pp. 645-655). Regarding FPR2, the role of the ALX/FPR2 receptor-ligand interaction in regulating dextran sulfate sodium (DDS)-induced colitis revealed that treatment with an ALX/FPR2 agonist, 15-epi-lipoxin A4, reverses the enhanced sensitivity of annexin A1 (−/−) mice to DDS-colitis (Babbin, B. A. et al., supra).

FPR1 is also functionally expressed on human lens epithelial cells and appears to have a direct functional role in lens development and maintenance (Schneider et al., J. Biol. Chem., V287, pp. 40779-40792, 2012).

We have discovered phenyl urea derivatives that exhibit selectivity for FPR1 relative to FPR2. To our knowledge, the present invention provides the first compounds to selectively modulate FPR1.

Other phenyl urea derivatives are known. For example:

Journal of Combinatorial Chemistry (2007), 9(3), 370-385 teaches a thymidinyl dipeptide urea library with structural similarity to the nucleoside peptide class of antibiotics:

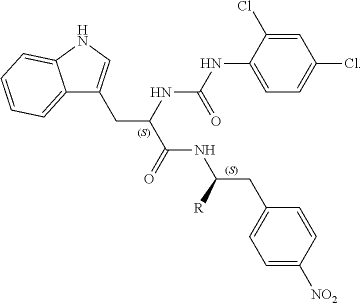

Helvetica Chimica Acta (1998), 81(7), 1254-1263 teaches the synthesis and spectroscopic characterization of 4-chlorophenyl isocyanate (1-chloro-4-isocyanatobenzene) adducts with amino acids as potential dosimeters for the biomonitoring of isocyanate exposure:

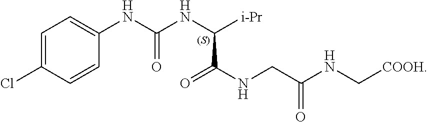

Yingyong Huaxue (1990), 7(1), 1-9 teaches the structure-activity relationships of di- and tripeptide sweeteners and of L-phenyl alanine derivatives:

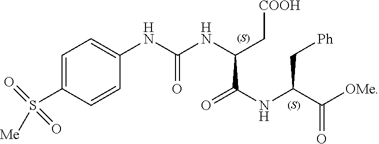

FR 2533210 discloses L-phenyl alanine derivatives as synthetic sweeteners:

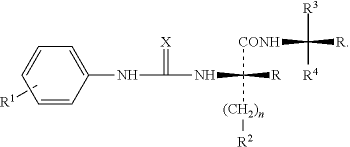

The following compounds are known as registered with Chemical Abstract Services (CAS), identified herein by structure and CAS registry number:

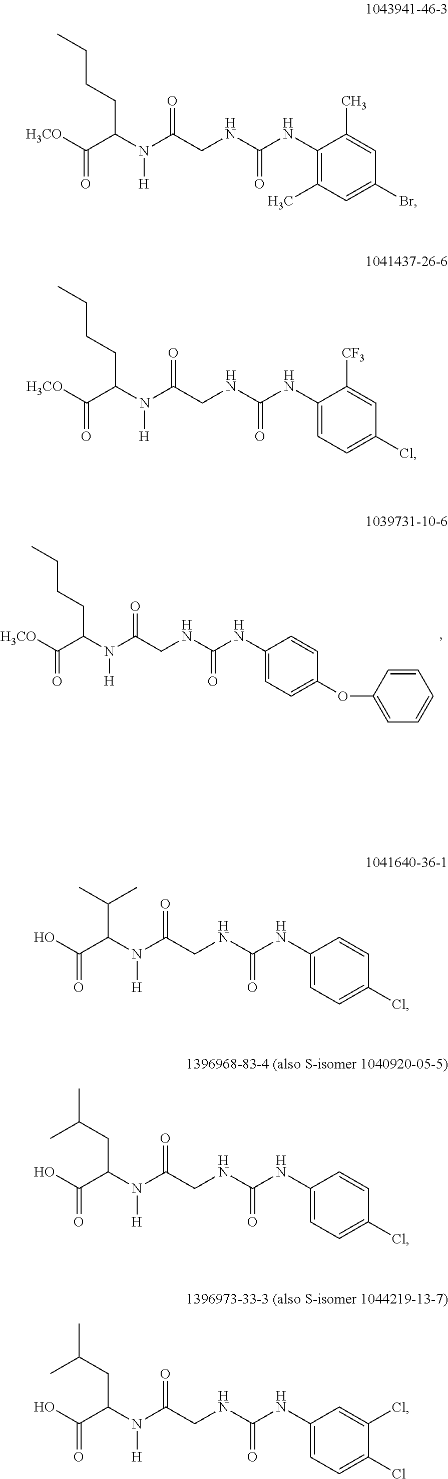 [SMILES: CCCCC(NC(=O)CNC(=O)Nc1c(C)cc(Br)cc1C)C(=O)OC ; CCCCC(NC(=O)CNC(=O)Nc1ccc(Cl)cc1C(F)(F)F)C(=O)OC ; CCCCC(NC(=O)CNC(=O)Nc1ccc(Oc2ccccc2)cc1)C(=O)OC ; CC(C)C(NC(=O)CNC(=O)Nc1ccc(Cl)cc1)C(=O)O ; CC(C)CC(NC(=O)CNC(=O)Nc1ccc(Cl)cc1)C(=O)O ; CC(C)CC(NC(=O)CNC(=O)Nc1ccc(Cl)c(Cl)c1)C(=O)O]

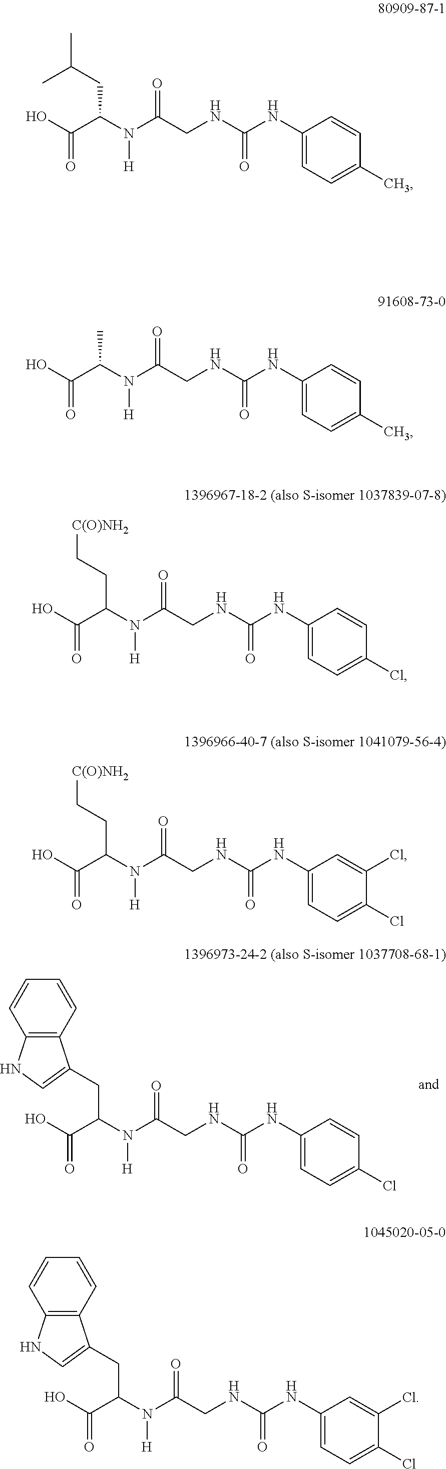

### SUMMARY OF THE INVENTION

A group of phenyl urea derivatives, which are potent and selective FPR1 modulators, has been discovered. As such, the compounds described herein are useful in treating a wide variety of disorders associated with inflammatory conditions modulated, at least in part, by the FPR receptor. The disorders may be associated with the modulation of FPR1 and/or FPR2, or with selective modulation of FPR1 relative to FPR2. The term “modulator” as used herein includes, but is not limited to: receptor agonist, antagonist, inverse agonist, inverse antagonist, partial agonist, and partial antagonist.

This invention describes compounds of Formula I, Ia, II and IIa, which modulate FPR biological activity. The compounds in accordance with the present invention are thus of use in medicine, for example, in the treatment of mammalian subjects, including humans, with diseases and/or conditions that are alleviated by FPR modulation (such as FPR1 and/or FPR2 agonism, or FPR1 agonism, or selective agonism of FPR1 relative to FPR2).

In one aspect, the invention provides a compound represented by Formula I:

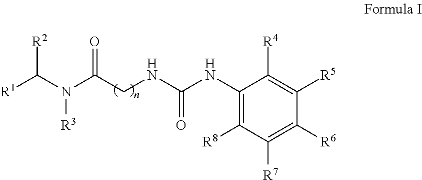

wherein:
R¹ is —COOH, —C(O)OR^{a}, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole; and R^{a} is optionally substituted C₁₋₆ alkyl, wherein said optional alkyl substituent is selected from OH, halogen, —OC₁₋₈ alkyl and —(O(CH₂)₁₋₈)_{q}—OC₁₋₈ alkyl; and q is 1, 2, 3, 4, 5 or 6;
R² is optionally substituted C₁₋₆ alkyl, wherein said optional alkyl substituent is selected from —OH, —SH, —OC₁₋₆ alkyl, —SC₁₋₆ alkyl, —COOH, —C(O)OC₁₋₆ alkyl, —C(O)NH₂, optionally substituted C₃₋₈ cycloalkyl, optionally substituted C₃₋₈ cycloalkenyl, optionally substituted C₆₋₁₀ aryl, and optionally substituted heterocycle;
R³ is H or unsubstituted C₁₋₆ alkyl;
R⁴ is H, optionally substituted C₁₋₆ alkyl, C₁₋₆ haloalkyl, optionally substituted C₃₋₈ cycloalkyl, optionally substituted C₃₋₈ cycloalkenyl, optionally substituted C₆₋₁₀ aryl, optionally substituted heterocycle, halogen, —NR¹¹R¹², —S(O)ₘR⁹, —C(O)R¹⁰, —C(O)OC₁₋₆ alkyl, —C(O)SC₁₋₆ alkyl, —OR¹³ or —SR¹³;
R⁵ is H, optionally substituted C₁₋₆ alkyl, C₁₋₆ haloalkyl, halogen, —S(O)ₘR⁹ or —C(O)R¹⁰;
R⁶ is optionally substituted C₁₋₆ alkyl, C₁₋₆ haloalkyl, optionally substituted C₃₋₈ cycloalkyl, optionally substituted C₃₋₈ cycloalkenyl, optionally substituted C₆₋₁₀ aryl, optionally substituted heterocycle, halogen, —S(O)ₘR⁹, —C(O)R¹⁰ or —OR¹¹;
R⁷ is H, optionally substituted C₁₋₆ alkyl, C₁₋₆ haloalkyl, halogen, —S(O)ₘR⁹ or —C(O)R¹⁰;
R⁸ is H, optionally substituted C₁₋₆ alkyl, C₁₋₆ haloalkyl, optionally substituted C₃₋₈ cycloalkyl, optionally substituted C₃₋₈ cycloalkenyl, optionally substituted C₆₋₁₀ aryl, optionally substituted heterocycle, halogen, —NR¹¹R¹², —S(O)ₘR⁹, —C(O)R¹⁰, —C(O)OC₁₋₆ alkyl, —C(O)SC₁₋₆ alkyl, —OR¹³ or —SR¹³;
each R⁹ is independently —OH, optionally substituted C₁₋₆ alkyl or optionally substituted C₆₋₁₀ aryl;
each R¹⁰ is independently —OH, optionally substituted C₁₋₆ alkyl or optionally substituted C₆₋₁₀ aryl;
each R¹¹ is independently H, optionally substituted C₁₋₈ alkyl, optionally substituted C₃₋₈ cycloalkyl, optionally substituted C₃₋₈ cycloalkenyl, optionally substituted C₆₋₁₀ aryl or optionally substituted heterocycle;
each R¹² is independently H, optionally substituted C₁₋₈ alkyl, optionally substituted C₃₋₈ cycloalkyl, optionally substituted C₃₋₈ cycloalkenyl, optionally substituted C₆₋₁₀ aryl or optionally substituted heterocycle;
each R¹³ is independently H or optionally substituted C₁₋₈ alkyl;
each m is independently 1 or 2; and
n is 1, 2 or 3;
or a single enantiomer thereof;
or a mixture of enantiomers thereof;
or a tautomer of the foregoing;
or pharmaceutically acceptable salt of the foregoing.
provided that the compound is not:

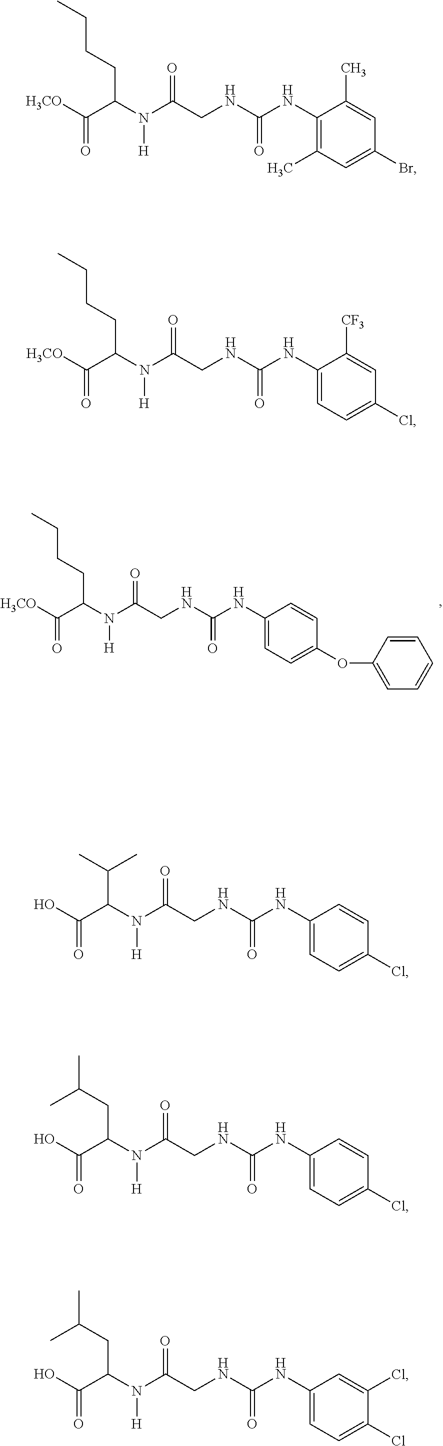 [SMILES: CCCCC(NC(=O)CNC(=O)Nc1c(C)cc(Br)cc1C)C(=O)OC ; CCCCC(NC(=O)CNC(=O)Nc1ccc(Cl)cc1C(F)(F)F)C(=O)OC ; CCCCC(NC(=O)CNC(=O)Nc1ccc(Oc2ccccc2)cc1)C(=O)OC ; CC(C)C(NC(=O)CNC(=O)Nc1ccc(Cl)cc1)C(=O)O ; CC(C)CC(NC(=O)CNC(=O)Nc1ccc(Cl)cc1)C(=O)O ; CC(C)CC(NC(=O)CNC(=O)Nc1ccc(Cl)c(Cl)c1)C(=O)O]

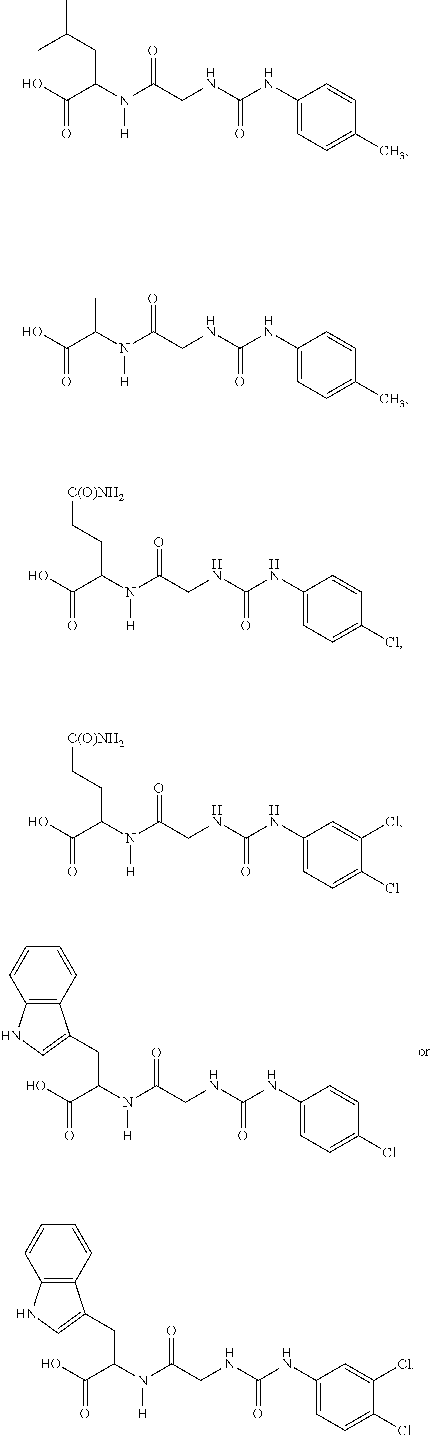

In another aspect, the invention provides a compound of Formula II:

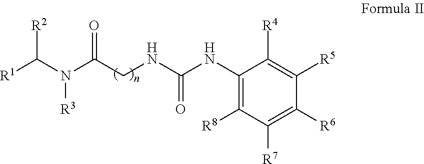

wherein:
R¹ is —COOH, —C(O)OR^{a}, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
   wherein R^{a} is optionally substituted C₁₋₆ alkyl, wherein said optional alkyl substituent is selected from OH, halogen, —OC₁₋₈ alkyl and —(O(CH₂)₁₋₈)_{q}—OC₁₋₈ alkyl; and q is 1, 2, 3, 4, 5 or 6;
R² is unsubstituted C₁₋₆ alkyl or benzyl;
R³ is H or unsubstituted C₁₋₆ alkyl;
R⁴ is H or halogen;
R⁵ is H;
R⁶ is C₁₋₆ haloalkyl or bromine;
R⁷ is H;
R⁸ is H or halogen; and
n is 1 or 2;
or a single enantiomer thereof;
or a mixture of enantiomers thereof;
or a tautomer of the foregoing;
or pharmaceutically acceptable salt of the foregoing.

In another aspect of the invention, there are provided pharmaceutical compositions comprising a therapeutically effective amount of at least one compound of the invention described herein in a pharmaceutically acceptable carrier.

In another aspect of the invention, there are provided compounds that selectively agonize FPR1 compared to FPR2. In further aspects, the compound shows at least 10-fold selectivity for FPR1 compared to FPR2, or at least 20-fold selectivity for FPR1 compared to FPR2. In yet further aspects, the compound shows at least 100-fold selectivity, at least 200-fold selectivity, or at least 300-fold selectivity for FPR1 compared to FPR2. In the preceding aspects, the selectivity is reported based on the ratio of the EC₅₀ for agonizing FPR2 to the EC₅₀ for agonizing FPR1.

In yet another aspect of the invention, there are provided methods for treating disorders associated with FPR modulation, such as FPR1 and/or FPR2 agonism, or selective agonism of FPR1 relative to FPR2. Such methods can be performed, for example, by administering to a subject in need thereof a pharmaceutical composition containing a therapeutically effective amount of at least one compound of the invention. In some aspects, the disorder is an inflammatory disease or condition. In further aspects, the inflammatory disease or condition is an ocular inflammatory disease or condition, such as dry eye or post-surgical inflammation, including post-cataract surgical inflammation. In yet further aspects, the inflammatory disease or condition is a dermal inflammatory disease or condition, such as psoriasis or rosacea. In further aspects, the method involves treating dermal wounds and promotes the healing of dermal wounds. In further aspects, the inflammatory disease or condition is a systemic inflammatory disease or condition. In yet further aspects, the disease or condition is an autoimmune disease or condition. In further aspects, the subject is a mammal, such as a human or non-human primate.

### DETAILED DESCRIPTION OF THE INVENTION

It is to be understood that both the foregoing general description and the following detailed description are exemplary and explanatory only and are not restrictive of the invention claimed. As used herein, the use of the singular includes the plural unless specifically stated otherwise.

The term “alkyl”, as used herein, refers to saturated, monovalent or divalent hydrocarbon moieties having linear or branched moieties or combinations thereof. Alkyl groups typically contain 1 to 6 carbon atoms (i.e., C₁₋₆ alkyl), but may contain a variable number of carbon atoms as specified. For example, an alkyl group may comprise 1 to 4 carbon atoms (i.e., C₁₋₄ alkyl), or 1 to 3 carbon atoms (i.e., C₁₋₃ alkyl). Alkyl groups are optionally substituted with one or more groups including, but not limited to: halogen, hydroxyl, thiol, cycloalkyl, heterocycle, aryl, ether, thioether, amine, nitro, nitrile, amide, sulfonamide, ester, thioester, aldehyde, carboxylic acid, ketone, sulfonic acid, phosphonic acid, and/or phosphoric acid. For example, substituted alkyl includes haloalkyl, such as perhaloalkyl or perfluoroalkyl (e.g., —CF₃). In a further example, substituted alkyl includes C, alkyl substituted with C₁₋₆ aryl (e.g., benzyl, which is (—CH₂-phenyl). One or more methylene (CH₂) groups of an alkyl can be replaced by oxygen, sulfur, —NH—, carbonyl, sulfoxide, sulfonyl, or by a divalent C₃₋₈ cycloalkyl; one or more methine (CH) groups of an alkyl can be replaced by nitrogen. Unsubstituted C₁₋₄ alkyl includes methyl, ethyl, n-propyl, isopropyl, n-butyl, isobutyl, sec-butyl and t-butyl. Unsubstituted C₁₋₃ alkyl includes methyl, ethyl, n-propyl and isopropyl.

The term “alkylene” as used herein refers to a bivalent saturated aliphatic radical derived from an alkene by opening of the double bond, or from an alkane by removal of two hydrogen atoms from one or from different carbon atoms. An alkylene may comprise 1 to 8 carbon atoms (i.e., C₁₋₈ alkylene), for example, a C₁ alkylene is methylene (—CH₂—); a C₂ alkylene is ethylene (—CH₂CH₂—), and so on.

The term “cycloalkyl”, as used herein, refers to a monovalent or divalent group of 3 to 8 carbon atoms (i.e., C₃₋₈ cycloalkyl) derived from a saturated cyclic hydrocarbon. Cycloalkyl groups can be monocyclic or polycyclic. Cycloalkyl groups are optionally substituted with one or more groups including, but not limited to: halogen, hydroxyl, thiol, alkyl, cycloalkyl, heterocycle, aryl, ether, thioether, amine, nitro, nitrile, amide, sulfonamide, ester, thioester, aldehyde, carboxylic acid, ketone, sulfonic acid, phosphonic acid, and/or phosphoric acid.

The term “cycloalkenyl”, as used herein, refers to a monovalent or divalent group of 3 to 8 carbon atoms (i.e., C₃₋₈ cycloalkenyl) derived from a saturated cycloalkyl having one or more double bonds. Cycloalkenyl groups can be monocyclic or polycyclic. Cycloalkenyl groups are optionally substituted by one or more groups including, but not limited to: halogen, hydroxyl, thiol, alkyl, cycloalkyl, heterocycle, aryl, ether, thioether, amine, nitro, nitrile, amide, sulfonamide, ester, thioester, aldehyde, carboxylic acid, ketone, sulfonic acid, phosphonic acid, and/or phosphoric acid.

The term “heterocycle” as used herein, refers to a 3 to 10 membered ring, which can be aromatic (i.e., a heteroaryl) or non-aromatic, saturated or unsaturated, containing at least one heteroatom selected from O, N and S, or combinations of at least two thereof, interrupting the carbocyclic ring structure. The heterocyclic ring can be interrupted by one or more C═O; the S and/or N heteroatom can be oxidized. Heterocycles can be monocyclic or polycyclic. Heterocyclic ring moieties are optionally substituted with one or more groups including, but not limited to: halogen, hydroxyl, thiol, alkyl, cycloalkyl, heterocycle, aryl, ether, thioether, amine, nitro, nitrile, amide, sulfonamide, ester, thioester, aldehyde, carboxylic acid, ketone, sulfonic acid, phosphonic acid, and/or phosphoric acid.

The term “aryl” as used herein, refers to an aromatic hydrocarbon ring containing 6 to 10 carbon atoms (i.e., C₆₋₁₀ aryl). Aryl groups are optionally substituted by one or more groups including, but not limited to: halogen, hydroxyl, alkyl, cycloalkyl, heterocycle, aryl, ether, amine, nitro, nitrile, amide, sulfonamide, ester, aldehyde, carboxylic acid, ketone, sulfonic acid, phosphonic acid, and/or phosphoric acid. Aryl can be monocyclic or polycyclic.

The term “halogen”, as used herein, refers to an atom of fluorine, chlorine, bromine, and/or iodine.

The term “amine” or “amino” as used herein, represents a group of formula “—NR^{x}R^{y}”, wherein R^{x} and R^{y} can be the same or independently H, alkyl, aryl, cycloalkyl, cycloalkenyl or heterocyclyl, as defined above.

The term “amide” as used herein, represents a group of formula “—C(O)N(R^{x})(R^{y})” or “—NR^{x}C(O)R^{y}” wherein R^{x} and R^{y} can be the same or independently H, alkyl, aryl, cycloalkyl, cycloalkenyl or heterocyclyl, as defined above.

The term “sulfonamide” as used herein, represents a group of formula “—S(O)₂N(R^{x})(R^{y})” or “—NR^{x}S(O)₂R^{y}” wherein R^{x} and R^{y} can be the same or independently H, alkyl, aryl, cycloalkyl, cycloalkenyl or heterocyclyl, as defined above.

The term “aldehyde” as used herein, represents a group of formula “—C(O)H”.

The term “ester” as used herein, represents a group of formula “—C(O)O(R^{x})”, wherein R^{x} is alkyl, aryl, cycloalkyl, cycloalkenyl or heterocyclyl, as defined above.

The term “thioester” as used herein, represents a group of formula “—C(O)S(R^{x})”, wherein R^{x} is alkyl, aryl, cycloalkyl, cycloalkenyl or heterocyclyl, as defined above.

The term “ketone” as used herein, represents a group of formula “—C(O)R^{x}” wherein R^{x} is alkyl, aryl, cycloalkyl, cycloalkenyl or heterocyclyl, as defined above.

The term “hydroxyl” as used herein, represents a group of formula “—OH”.

The term “thiol” as used herein, represents a group of formula “—SH”.

The term “carbonyl” as used herein, represents a group of formula “—C(O)—”.

The term “carboxyl” as used herein, represents a group of formula “—C(O)O—”.

The term “carboxylic acid” as used herein, represents a group of formula “—C(O)OH”.

The term “carboxylate” as used herein, represents a group of formula “—C(O)O⁻”.

The term “sulfoxide” as used herein, represents a group of formula “—S(O)—”.

The term “sulfonyl” as used herein, represents a group of formula “—SO₂—”.

The term “sulfate” as used herein, represents a group of formula “—OS(O)₂O⁻”.

The term “sulphonic acid” as used herein, represents a group of formula “—S(O)₂OH”.

The term “phosphonic acid” as used herein, represents a group of formula “—P(O)(OH)₂”.

The term “phosphoric acid” as used herein, represents a group of formula “—(O)P(O)(OH)₂”.

The term “nitro” as used herein, represents a group of formula “—NO₂”.

The term “nitrile” as used herein, represents a group of formula “—CN”.

The term “ether” as used herein, represents a group of formula “—OR^{x}”, wherein R^{x} is alkyl, aryl, cycloalkyl, cycloalkenyl or heterocyclyl, as defined above.

The term “thioether” as used herein, represents a group of formula “—SR^{x}”, wherein R^{x} is alkyl, aryl, cycloalkyl, cycloalkenyl or heterocyclyl, as defined above.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “pharmaceutically acceptable salts” refers to salts or complexes that retain the desired biological activity of compounds of the invention, and exhibit minimal or no undesired toxicological effects. The “pharmaceutically acceptable salts” according to the invention include therapeutically active, non-toxic base or acid salt forms, which the compounds of Formula I, Ia, II and IIa are able to form.

The term “carboxylate isostere”, as used herein, refers to a group that replaces a carboxylic acid, such as a group selected from sulfonate, sulfonic acid, phosphonate, phosphonic acid, phosphoric acid, boronic acid and unsubstituted or substituted heterocycle, wherein said heterocycle is selected from tetrazole, imidazole, thiazole, oxazole, isoxazole, oxadiazole, thiadiazole, triazole, thiophene, pyrazole and pyrole; and wherein said heterocycle substituent is selected from unsubstituted and substituted C₁₋₈ alkyl, wherein said alkyl substituent is selected from OH and halogen.

The term “therapeutically effective amount” means the amount of a pharmaceutical composition that will elicit a biological or medical response in a subject in need thereof that is being sought by the researcher, veterinarian, medical doctor or other clinician.

In one embodiment, the present invention provides a compound represented by Formula I:

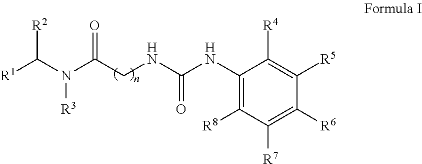

wherein:
R¹ is —COOH, —C(O)OR^{a}, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole; and R^{a} is optionally substituted C₁₋₆ alkyl, wherein said alkyl optional substituent is selected from OH, halogen, —OC₁₋₈ alkyl and —(O(CH₂)₁₋₈)_{q}—OC₁₋₈ alkyl; and q is 1, 2, 3, 4, 5 or 6;
R² is optionally substituted C₁₋₆ alkyl, wherein said optional alkyl substituent is selected from —OH, —SH, —OC₁₋₆ alkyl, —SC₁₋₆ alkyl, —COOH, —C(O)OC₁₋₆ alkyl, —C(O)NH₂, optionally substituted C₃₋₈ cycloalkyl, optionally substituted C₃₋₈ cycloalkenyl, optionally substituted C₆₋₁₀ aryl, and optionally substituted heterocycle;
R³ is H or unsubstituted C₁₋₆ alkyl;
R⁴ is H, optionally substituted C₁₋₆ alkyl, C₁₋₆ haloalkyl, optionally substituted C₃₋₈ cycloalkyl, optionally substituted C₃₋₈ cycloalkenyl, optionally substituted C₆₋₁₀ aryl, optionally substituted heterocycle, halogen, —NR¹¹R¹², —S(O)ₘR⁹, —C(O)R¹⁰, —C(O)OC₁₋₆ alkyl, —C(O)SC₁₋₆ alkyl, —OR¹³ or —SR¹³;
R⁵ is H, optionally substituted C₁₋₆ alkyl, C₁₋₆ haloalkyl, halogen, —S(O)ₘR⁹ or —C(O)R¹⁰;
R⁶ is optionally substituted C₁₋₆ alkyl, C₁₋₆ haloalkyl, optionally substituted C₃₋₈ cycloalkyl, optionally substituted C₃₋₈ cycloalkenyl, optionally substituted C₆₋₁₀ aryl, optionally substituted heterocycle, halogen, —S(O)ₘR⁹, —C(O)R¹⁰ or —OR¹¹;
R⁷ is H, optionally substituted C₁₋₆ alkyl, C₁₋₆ haloalkyl, halogen, —S(O)ₘR⁹ or —C(O)R¹⁰;
R⁸ is H, optionally substituted C₁₋₆ alkyl, C₁₋₆ haloalkyl, optionally substituted C₃₋₈ cycloalkyl, optionally substituted C₃₋₈ cycloalkenyl, optionally substituted C₆₋₁₀ aryl, optionally substituted heterocycle, halogen, —NR¹¹R¹², —S(O)ₘR⁹, —C(O)R¹⁰, —C(O)OC₁₋₆ alkyl, —C(O)SC₁₋₆ alkyl, —OR¹³ or —SR¹³;
each R⁹ is independently —OH, optionally substituted C₁₋₆ alkyl or optionally substituted C₆₋₁₀ aryl;
each R¹⁰ is independently —OH, optionally substituted C₁₋₆ alkyl or optionally substituted C₆₋₁₀ aryl;
each R¹¹ is independently H, optionally substituted C₁₋₈ alkyl, optionally substituted C₃₋₈ cycloalkyl, optionally substituted C₃₋₈ cycloalkenyl, optionally substituted C₆₋₁₀ aryl or optionally substituted heterocycle;
each R¹² is independently H, optionally substituted C₁₋₈ alkyl, optionally substituted C₃₋₈ cycloalkyl, optionally substituted C₃₋₈ cycloalkenyl, optionally substituted C₆₋₁₀ aryl or optionally substituted heterocycle;
each R¹³ is independently H or optionally substituted C₁₋₈ alkyl;
each m is independently 1 or 2; and
n is 1, 2 or 3;
or a single enantiomer thereof;
or a mixture of enantiomers thereof;
or a tautomer of the foregoing;
or pharmaceutically acceptable salt of the foregoing.
provided that the compound is not:

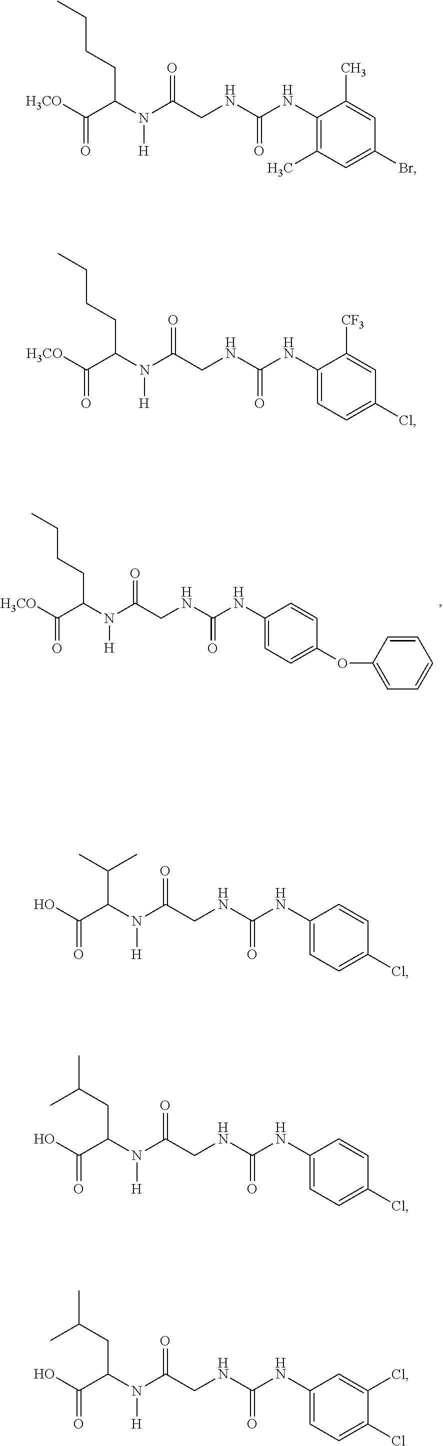 [SMILES: CCCCC(NC(=O)CNC(=O)Nc1c(C)cc(Br)cc1C)C(=O)OC ; CCCCC(NC(=O)CNC(=O)Nc1ccc(Cl)cc1C(F)(F)F)C(=O)OC ; CCCCC(NC(=O)CNC(=O)Nc1ccc(Oc2ccccc2)cc1)C(=O)OC ; CC(C)C(NC(=O)CNC(=O)Nc1ccc(Cl)cc1)C(=O)O ; CC(C)CC(NC(=O)CNC(=O)Nc1ccc(Cl)cc1)C(=O)O ; CC(C)CC(NC(=O)CNC(=O)Nc1ccc(Cl)c(Cl)c1)C(=O)O]

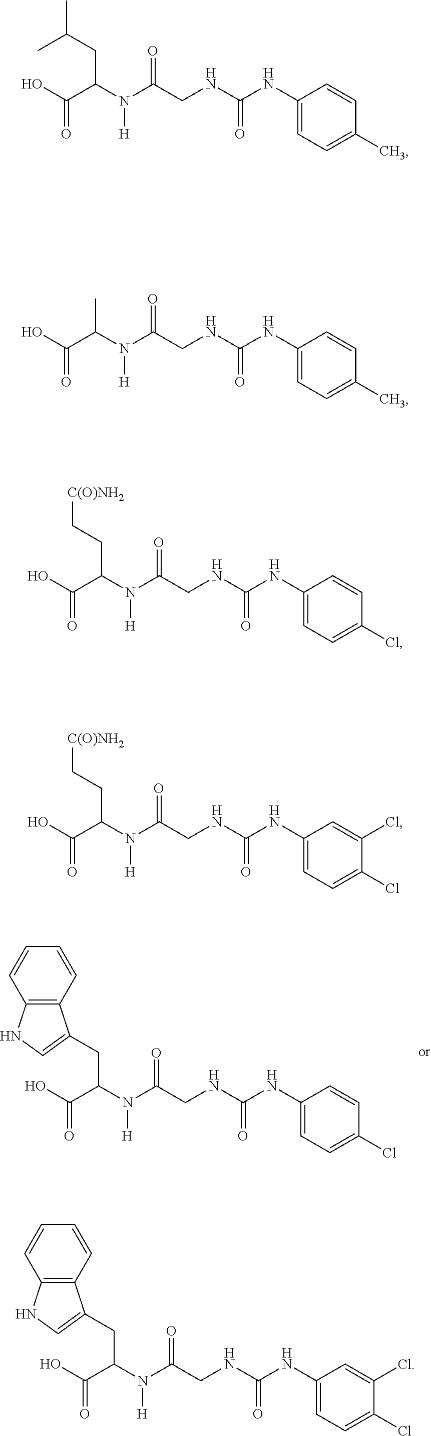

In some embodiments, there are provided compounds of Formula I, wherein R¹ is —COOH or —C(O)OR^{a}, wherein R^{a} is unsubstituted or substituted C₁₋₆ alkyl; wherein said alkyl substituent is selected from OH, halogen, —OC₁₋₈ alkyl and —(O(CH₂)₁₋₈)_{q}—OC₁₋₈ alkyl; and q is 1, 2, 3, 4, 5 or 6. In further embodiments, there are provided compound of Formula I, wherein R¹ is —COOH or —C(O)OR^{a}, and R^{a} is unsubstituted C₁₋₆ alkyl. In other embodiments, there are provided compounds of Formula I, wherein R¹ is —COOH.

In some embodiments, there are provided compounds of Formula I, wherein R¹ is —COOH, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole.

In some embodiments, there are provided compounds of Formula I wherein R² is a sidechain derived from a naturally occurring amino acid, such as glycine, alanine, valine, leucine, isoleucine, serine, cysteine, threonine, methionine, asparagine, glutamine, aspartic acid, glutamic acid, lysine, arginine, phenylalanine, tyrosine, tryptophan or histidine. In some embodiments, R² is —CH₃, —CH(CH₃)₂, —CH₂CH(CH₃)₂, —CH(CH₃)CH₂CH₃, —CH₂OH, —CH(OH)CH₃, —CH₂SH, —CH₂CH₂SCH₃, —CH₂C(O)NH₂, —CH₂CH₂C(O)NH₂, —CH₂C(O)OH, —CH₂CH₂C(O)OH, —CH₂CH₂CH₂CH₂NH₂, —CH₂CH₂CH₂NHC(NH)NH₂,

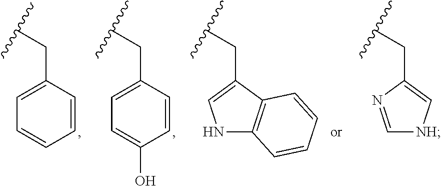

or a salt thereof.

In some embodiments, there are provided compounds of Formula I, wherein R² is unsubstituted C₁₋₆ alkyl or benzyl. In some embodiments, R² is unsubstituted C₁₋₆ alkyl. In other embodiments, R² is unsubstituted C₁₋₄ alkyl, such as methyl, ethyl, n-propyl, isopropyl, n-butyl, tert-butyl, isobutyl or sec-butyl. In another embodiment, R² is unsubstituted C₁₋₃ alkyl, such as methyl, ethyl, n-propyl or isopropyl. In other embodiments, R² is unsubstituted benzyl.

In some embodiments, there are provided compounds of Formula I, wherein R³ is H or unsubstituted C₁₋₃ alkyl. In some embodiments, R³ is H. In another embodiment, R³ is unsubstituted C₁₋₃ alkyl, such as methyl, ethyl, n-propyl or isopropyl. In yet another embodiment, R³ is methyl.

In some embodiments, there are provided compounds of Formula I, wherein n is 1 or 2. In another embodiment, n is 1. In yet other embodiment, n is 2.

In some embodiments, there are provided compounds of Formula I, wherein R⁴ is H, F or C₁₋₆ haloalkyl; R⁵ is H, F or C₁₋₆ haloalkyl; R⁷ is H, F or C₁₋₆ haloalkyl; and R⁸ is H, F or C₁₋₆ haloalkyl. In another embodiment, there are provided compounds wherein R⁴ is H, F or C₁₋₆ fluoroalkyl; R⁵ is H, F or C₁₋₆ fluoroalkyl; R⁷ is H, F or C₁₋₆ fluoroalkyl; and R⁸ is H, F or C₁₋₆ fluoroalkyl. In a further embodiment, there are provided compounds wherein R⁴ is H, F or C₁₋₆ perfluoroalkyl; R⁵ is H, F or C₁₋₆ perfluoroalkyl; R⁷ is H, F or C₁₋₆ perfluoroalkyl; and R⁸ is H, F or C₁₋₆ perfluoroalkyl. In yet a further embodiment, there are provided compounds of Formula I wherein R⁴ is H, F or CF₃; R⁵ is H, F or CF₃; R⁷ is H, F or CF₃; and R⁸ is H, F or CF₃. In another embodiment, R⁴ is H or F; R⁵ is H or F; R⁷ is H or F; and R⁸ is H or F. In a further embodiment, there are provided compounds of Formula I, wherein at least one of R⁴ and R⁸ is H. In other embodiment, there are provided compounds of Formula I, wherein each of R⁴, R⁵, R⁷ and R⁸ is H.

In some embodiments, there are provided compounds of Formula I, wherein R⁶ is C₁₋₆ haloalkyl or halogen. In one embodiment, R⁶ is C₁₋₆ haloalkyl or Br. In another embodiment, R⁶ is C₁₋₆ fluoroalkyl or bromine. In a further embodiment, R⁶ is C₁₋₆ perfluoroalkyl or bromine. In yet a further embodiment, R⁶ is CF₃ or bromine. In another embodiment, R⁶ is —CF₃. In another embodiment, R⁶ is —Br.

In some embodiments, there are provided compounds of Formula I, provided that R⁶ is not chlorine, methyl or —O—C₆₋₁₀aryl.

In some embodiments, there are provided compounds of Formula I, wherein each C₁₋₆ haloalkyl is independently replaced with C₁₋₆ perfluoroalkyl. In another embodiment, there are provided compounds of Formula I, wherein each C₁₋₆ haloalkyl is independently replaced with C₁₋₃ haloalkyl. In another embodiment, there are provided compounds of Formula I, wherein each C₁₋₆ haloalkyl is independently replaced with C₁₋₃ perfluoroalkyl. In another embodiment, there are provided compounds of Formula I, wherein each C₁₋₆ haloalkyl is independently replaced with —CF₃.

In another embodiment, there are provided compounds of Formula I, wherein:
R¹ is —COOH, —C(O)OR^{a}, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
   wherein R^{a} is optionally substituted C₁₋₆ alkyl, wherein said alkyl substituent is selected from OH, halogen, —OC₁₋₈ alkyl and —(O(CH₂)₁₋₈)_{q}—OC₁₋₈ alkyl; and q is 1, 2, 3, 4, 5 or 6;
R² is unsubstituted C₁₋₆ alkyl or benzyl;
R³ is H or unsubstituted C₁₋₆ alkyl;
R⁴ is H or F;
R⁵ is H;
R⁶ is C₁₋₆ fluoroalkyl or bromine;
R⁷ is H;
R⁸ is H or F; and
n is 1 or 2;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole; wherein R^{a} is unsubstituted C₁₋₆ alkyl;
R² is unsubstituted C₁₋₆ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole; wherein R^{a} is unsubstituted C₁₋₆ alkyl;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, thiophene, pyrazole and pyrrole; wherein R^{a} is unsubstituted C₁₋₆ alkyl;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole; wherein R^{a} is unsubstituted C₁₋₆ alkyl;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 2;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₆ alkyl or benzyl;
R³ is H or unsubstituted C₁₋₆ alkyl;
R⁴ is H or F;
R⁵ is H;
R⁶ is C₁₋₆ fluoroalkyl or bromine;
R⁷ is H;
R⁸ is H or F; and
n is 1 or 2;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₆ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a tautomer thereof;
or pharmaceutically acceptable salt of any one of the foregoing.

In another embodiment, there are provided compounds of Formula I, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, thiophene, pyrazole and pyrrole;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 2;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH or —C(O)OR^{a}, wherein R^{a} is unsubstituted C₁₋₆ alkyl;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1 or 2;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH or —C(O)OR^{a}, wherein R^{a} is unsubstituted C₁₋₆ alkyl;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH or —C(O)OR^{a}, wherein R^{a} is unsubstituted C₁₋₆ alkyl;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 2;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1 or 2;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 2;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH;
R² is unsubstituted C₁₋₃ alkyl or benzyl;
R³ is H;
R⁴ is H;
R⁵ is H;
R⁶ is —CF₃ or bromine;
R⁷ is H;
R⁸ is H; and
n is 1;
or a single enantiomer thereof;
or a mixture of enantiomers thereof;
or a tautomer of the foregoing;
or pharmaceutically acceptable salt of the foregoing.

In another embodiment, there are provided compounds of Formula I, wherein:
R¹ is —COOH;
R² is unsubstituted C₁₋₃ alkyl or benzyl;
R³ is H;
R⁴ is H;
R⁵ is H;
R⁶ is —CF₃ or bromine;
R⁷ is H;
R⁸ is H; and
n is 2;
or a single enantiomer thereof;
or a mixture of enantiomers thereof;
or a tautomer of the foregoing;
or pharmaceutically acceptable salt of the foregoing.

In some embodiments, provided herein are compounds of Formula Ia:

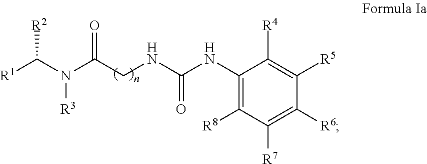

wherein the compound has the specific stereochemistry shown at the carbon bearing R², and wherein:
R¹ is —COOH, —C(O)OR^{a}, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole; and R^{a} is optionally substituted C₁₋₆ alkyl, wherein said alkyl substituent is selected from OH, halogen, —OC₁₋₈ alkyl and —(O(CH₂)₁₋₈)_{q}—OC₁₋₈ alkyl; and q is 1, 2, 3, 4, 5 or 6;
R² is optionally substituted C₁₋₆ alkyl, wherein said optional alkyl substituent is selected from —OH, —SH, —OC₁₋₆ alkyl, —SC₁₋₆ alkyl, —COOH, —C(O)OC₁₋₆ alkyl, —C(O)NH₂, optionally substituted C₃₋₈ cycloalkyl, optionally substituted C₃₋₈ cycloalkenyl, optionally substituted C₆₋₁₀ aryl, and optionally substituted heterocycle;
R³ is H or unsubstituted C₁₋₆ alkyl;
R⁴ is H, optionally substituted C₁₋₆ alkyl, C₁₋₆ haloalkyl, optionally substituted C₃₋₈ cycloalkyl, optionally substituted C₃₋₈ cycloalkenyl, optionally substituted C₆₋₁₀ aryl, optionally substituted heterocycle, halogen, —NR¹¹R¹², —S(O)ₘR⁹, —C(O)R¹⁰, —C(O)OC₁₋₆ alkyl, —C(O)SC₁₋₆ alkyl, —OR¹³ or —SR¹³;
R⁵ is H, optionally substituted C₁₋₆ alkyl, C₁₋₆ haloalkyl, halogen, —S(O)ₘR⁹ or —C(O)R¹⁰;
R⁶ is optionally substituted C₁₋₆ alkyl, C₁₋₆ haloalkyl, optionally substituted C₃₋₈ cycloalkyl, optionally substituted C₃₋₈ cycloalkenyl, optionally substituted C₆₋₁₀ aryl, optionally substituted heterocycle, halogen, —S(O)ₘR⁹, —C(O)R¹⁰ or —OR¹¹;
R⁷ is H, optionally substituted C₁₋₆ alkyl, C₁₋₆ haloalkyl, halogen, —S(O)ₘR⁹ or —C(O)R¹⁰;
R⁸ is H, optionally substituted C₁₋₆ alkyl, C₁₋₆ haloalkyl, optionally substituted C₃₋₈ cycloalkyl, optionally substituted C₃₋₈ cycloalkenyl, optionally substituted C₆₋₁₀ aryl, optionally substituted heterocycle, halogen, —NR¹¹R¹², —S(O)ₘR⁹, —C(O)R¹⁰, —C(O)OC₁₋₆ alkyl, —C(O)SC₁₋₆ alkyl, —OR¹³ or —SR¹³;
is H, optionally substituted C₁₋₆ alkyl, C₁₋₆ haloalkyl, optionally substituted C₃₋₈ cycloalkyl, optionally substituted C₃₋₈ cycloalkenyl, optionally substituted C₆₋₁₀ aryl, optionally substituted heterocycle, halogen, NR¹¹R¹², —S(O)ₘR⁹, —C(O)R¹⁰ or —OR¹¹;
each R⁹ is independently —OH, optionally substituted C₁₋₆ alkyl or optionally substituted C₆₋₁₀ aryl;
each R¹⁰ is independently —OH, optionally substituted C₁₋₆ alkyl or optionally substituted C₆₋₁₀ aryl;
each R¹¹ is independently H, optionally substituted C₁₋₈ alkyl, optionally substituted C₃₋₈ cycloalkyl, optionally substituted C₃₋₈ cycloalkenyl, optionally substituted C₆₋₁₀ aryl or optionally substituted heterocycle;
each R¹² is independently H, optionally substituted C₁₋₈ alkyl, optionally substituted C₃₋₈ cycloalkyl, optionally substituted C₃₋₈ cycloalkenyl, optionally substituted C₆₋₁₀ aryl or optionally substituted heterocycle;
each R¹³ is independently H or optionally substituted C₁₋₈ alkyl;
each m is independently 1 or 2; and
n is 1, 2 or 3;
or a tautomer thereof;
or pharmaceutically acceptable salt of the foregoing;
provided that the compound is not:

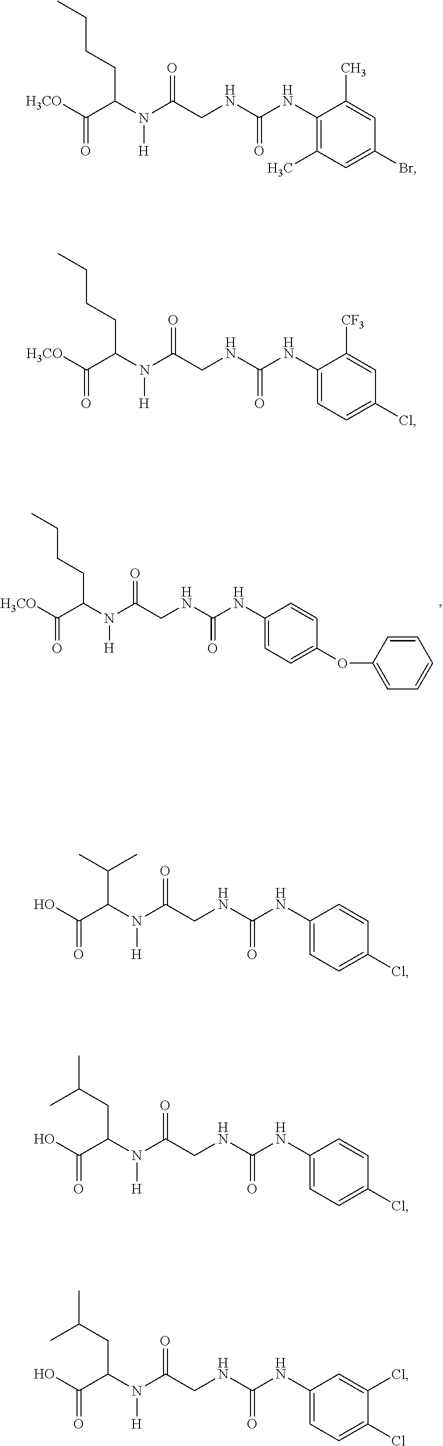 [SMILES: CCCCC(NC(=O)CNC(=O)Nc1c(C)cc(Br)cc1C)C(=O)OC ; CCCCC(NC(=O)CNC(=O)Nc1ccc(Cl)cc1C(F)(F)F)C(=O)OC ; CCCCC(NC(=O)CNC(=O)Nc1ccc(Oc2ccccc2)cc1)C(=O)OC ; CC(C)C(NC(=O)CNC(=O)Nc1ccc(Cl)cc1)C(=O)O ; CC(C)CC(NC(=O)CNC(=O)Nc1ccc(Cl)cc1)C(=O)O ; CC(C)CC(NC(=O)CNC(=O)Nc1ccc(Cl)c(Cl)c1)C(=O)O]

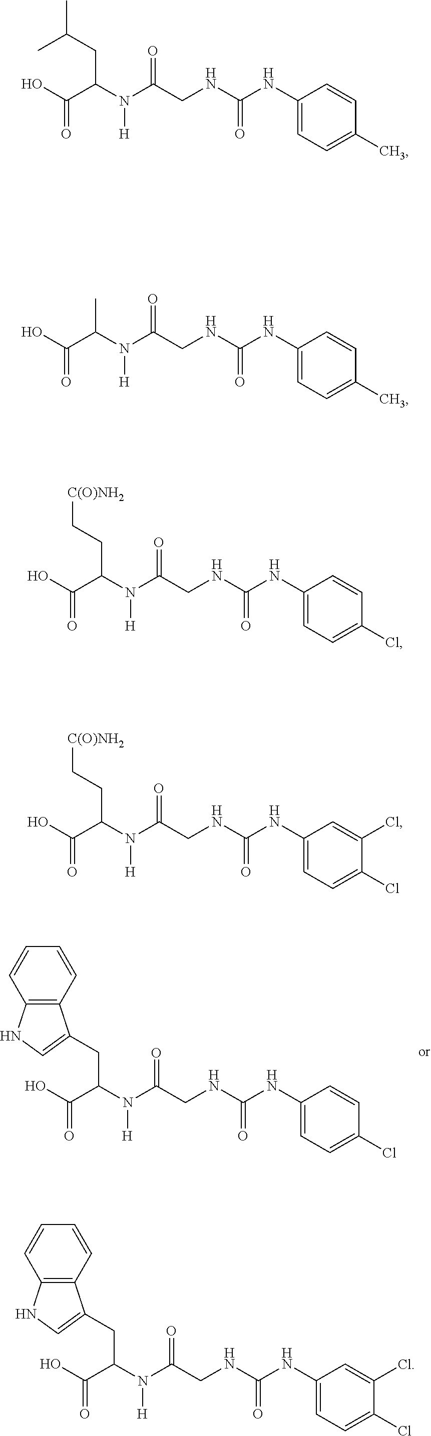

In some embodiments, there are provided compounds of Formula Ia, wherein R¹ is —COOH or —C(O)OR^{a}, wherein R^{a} is unsubstituted or substituted C₁₋₆ alkyl; wherein said alkyl substituent is selected from OH, halogen, —OC₁₋₈ alkyl and —(O(CH₂)₁₋₈)_{q}—OC₁₋₈ alkyl; and q is 1, 2, 3, 4, 5 or 6. In further embodiments, there are provided compound of Formula Ia, wherein R¹ is —COOH or —C(O)OR^{a}, and R^{a} is unsubstituted C₁₋₆ alkyl. In other embodiments, there are provided compounds of Formula Ia, wherein R¹ is —COOH.

In some embodiments, there are provided compounds of Formula Ia, wherein R¹ is —COOH, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole.

In some embodiments, there are provided compounds of Formula Ia, wherein R² is unsubstituted C₁₋₆ alkyl or benzyl. In some embodiments, R² is unsubstituted C₁₋₆ alkyl. In some embodiments, R² is unsubstituted C₁₋₄ alkyl, such as methyl, ethyl, n-propyl, isopropyl, n-butyl, tert-butyl, isobutyl or sec-butyl. In other embodiments, R² is unsubstituted benzyl.

In some embodiments, there are provided compounds of Formula Ia, wherein R³ is H or unsubstituted C₁₋₃ alkyl. In some embodiments, R³ is H. In another embodiment, R³ is unsubstituted C₁₋₃ alkyl, such as methyl, ethyl, n-propyl or isopropyl. In yet another embodiment, R³ is methyl.

In some embodiments, there are provided compounds of Formula Ia, wherein n is 1 or 2. In another embodiment, n is 1. In yet other embodiment, n is 2.

In some embodiments, there are provided compounds of Formula Ia, wherein R⁴ is H, F or C₁₋₆ haloalkyl; R⁵ is H, F or C₁₋₆ haloalkyl; R⁷ is H, F or C₁₋₆ haloalkyl; and R⁸ is H, F or C₁₋₆ haloalkyl. In another embodiment, there are provided compounds wherein R⁴ is H, F or C₁₋₆ fluoroalkyl; R⁵ is H, F or C₁₋₆ fluoroalkyl; R⁷ is H, F or C₁₋₆ fluoroalkyl; and R⁸ is H, F or C₁₋₆ fluoroalkyl. In a further embodiment, there are provided compounds of Formula Ia wherein R⁴ is H, F or C₁₋₆ perfluoroalkyl; R⁵ is H, F or C₁₋₆ perfluoroalkyl; R⁷ is H, F or C₁₋₆ perfluoroalkyl; and R⁸ is H, F or C₁₋₆ perfluoroalkyl. In yet a further embodiment, there are provided compounds of Formula Ia wherein R⁴ is H, F or CF₃; R⁵ is H, F or CF₃; R⁷ is H, F or CF₃; and R⁸ is H, F or CF₃. In another embodiment, R⁴ is H or F; R⁵ is H or F; R⁷ is H or F; and R⁸ is H or F. In a further embodiment, there are provided compounds of Formula Ia, wherein at least one of R⁴ and R⁸ is H. In other embodiment, there are provided compounds of Formula Ia, wherein each of R⁴, R⁵, R⁷ and R⁸ is H.

In some embodiments, there are provided compounds of Formula Ia, wherein R⁶ is C₁₋₆ haloalkyl or halogen. In one embodiment, R⁶ is C₁₋₆ haloalkyl or Br. In another embodiment, R⁶ is C₁₋₆ fluoroalkyl or bromine. In a further embodiment, R⁶ is C₁₋₆ perfluoroalkyl or bromine. In yet a further embodiment, R⁶ is CF₃ or bromine. In another embodiment, R⁶ is —CF₃. In another embodiment, R⁶ is —Br.

In some embodiments, there are provided compounds of Formula Ia, provided that R⁶ is not chlorine, methyl or —O—C₆₋₁₀aryl.

In some embodiments, there are provided compounds of Formula Ia, wherein each C₁₋₆ haloalkyl is independently replaced with C₁₋₆ perfluoroalkyl. In another embodiment, there are provided compounds of Formula Ia, wherein each C₁₋₆ haloalkyl is independently replaced with C₁₋₃ haloalkyl. In another embodiment, there are provided compounds of Formula Ia, wherein each C₁₋₆ haloalkyl is independently replaced with C₁₋₃ perfluoroalkyl. In another embodiment, there are provided compounds of Formula Ia, wherein each C₁₋₆ haloalkyl is independently replaced with —CF₃.

In another embodiment, there are provided compounds of Formula Ia, wherein:
R¹ is —COOH, —C(O)OR^{a}, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
   wherein R^{a} is optionally substituted C₁₋₆ alkyl, wherein said alkyl substituent is selected from OH, halogen, —OC₁₋₈ alkyl and —(O(CH₂)₁₋₈)_{q}—OC₁₋₈ alkyl; and q is 1, 2, 3, 4, 5 or 6;
R² is unsubstituted C₁₋₆ alkyl or benzyl;
R³ is H or unsubstituted C₁₋₆ alkyl;
R⁴ is H or F;
R⁵ is H;
R⁶ is C₁₋₆ fluoroalkyl or bromine;
R⁷ is H;
R⁸ is H or F; and
n is 1 or 2;
or a tautomer thereof;
or pharmaceutically acceptable salt of the foregoing.

In another embodiment, there are provided compounds of Formula Ia, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole; wherein R^{a} is unsubstituted C₁₋₆ alkyl;
R² is unsubstituted C₁₋₆ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a tautomer thereof;
or pharmaceutically acceptable salt of any one of the foregoing.

In another embodiment, there are provided compounds of Formula Ia, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole; wherein R^{a} is unsubstituted C₁₋₆ alkyl;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a tautomer thereof;
or pharmaceutically acceptable salt of the foregoing.

In another embodiment, there are provided compounds of Formula Ia, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, thiophene, pyrazole and pyrrole; wherein R^{a} is unsubstituted C₁₋₆ alkyl;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1;
or a tautomer thereof;
or pharmaceutically acceptable salt of the foregoing.

In another embodiment, there are provided compounds of Formula Ia, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole; wherein R^{a} is unsubstituted C₁₋₆ alkyl;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 2;
or a tautomer thereof;
or pharmaceutically acceptable salt of the foregoing.

In another embodiment, there are provided compounds of Formula Ia, wherein:
R¹ is —COOH, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₆ alkyl or benzyl;
R³ is H or unsubstituted C₁₋₆ alkyl;
R⁴ is H or F;
R⁵ is H;
R⁶ is C₁₋₆ fluoroalkyl or bromine;
R⁷ is H;
R⁸ is H or F; and
n is 1 or 2;
or a tautomer thereof;
or pharmaceutically acceptable salt of the foregoing.

In another embodiment, there are provided compounds of Formula Ia, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₆ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a tautomer thereof;
or pharmaceutically acceptable salt of any one of the foregoing.

In another embodiment, there are provided compounds of Formula Ia, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a tautomer thereof;
or pharmaceutically acceptable salt of the foregoing.

In another embodiment, there are provided compounds of Formula Ia, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, thiophene, pyrazole and pyrrole;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1;
or a tautomer thereof;
or pharmaceutically acceptable salt of the foregoing.

In another embodiment, there are provided compounds of Formula Ia, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 2;
or a tautomer thereof;
or pharmaceutically acceptable salt of the foregoing.

In another embodiment, there are provided compounds of Formula Ia, wherein:
R¹ is —COOH or —C(O)OR^{a}, wherein R^{a} is unsubstituted C₁₋₆ alkyl;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1 or 2;
or a tautomer thereof;
or pharmaceutically acceptable salt of the foregoing.

In another embodiment, there are provided compounds of Formula Ia, wherein:
R¹ is —COOH or —C(O)OR^{a}, wherein R^{a} is unsubstituted C₁₋₆ alkyl;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1;
or a tautomer thereof;
or pharmaceutically acceptable salt of the foregoing.

In another embodiment, there are provided compounds of Formula Ia, wherein:
R¹ is —COOH or —C(O)OR^{a}, wherein R^{a} is unsubstituted C₁₋₆ alkyl;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 2;
or a tautomer thereof;
or pharmaceutically acceptable salt of the foregoing.

In another embodiment, there are provided compounds of Formula Ia, wherein:
R¹ is —COOH;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1 or 2;
or a tautomer thereof;
or pharmaceutically acceptable salt of the foregoing.

In another embodiment, there are provided compounds of Formula Ia, wherein:
R¹ is —COOH;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1;
or a tautomer thereof;
or pharmaceutically acceptable salt of the foregoing.

In another embodiment, there are provided compounds of Formula Ia, wherein:
R¹ is —COOH;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 2;
or a tautomer thereof;
or pharmaceutically acceptable salt of the foregoing.

In another embodiment, there are provided compounds of Formula Ia, wherein:
R¹ is —COOH;
R² is unsubstituted C₁₋₃ alkyl or benzyl;
R³ is H;
R⁴ is H;
R⁵ is H;
R⁶ is —CF₃ or bromine;
R⁷ is H;
R⁸ is H; and
n is 1;
or a tautomer thereof;
or pharmaceutically acceptable salt of the foregoing.

In another embodiment, there are provided compounds of Formula Ia, wherein:
R¹ is —COOH;
R² is unsubstituted C₁₋₃ alkyl or benzyl;
R³ is H;
R⁴ is H;
R⁵ is H;
R⁶ is —CF₃ or bromine;
R⁷ is H;
R⁸ is H; and
n is 2;
or a tautomer thereof;
or pharmaceutically acceptable salt of the foregoing.

In another embodiment, the invention provides for compounds of Formula II:

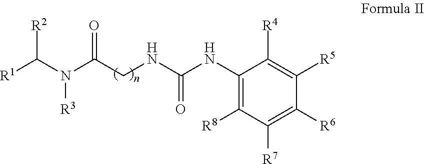

wherein:
R¹ is —COOH, —C(O)OR^{a}, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
   wherein R^{a} is optionally substituted C₁₋₆ alkyl, wherein said alkyl substituent is selected from OH, halogen, —OC₁₋₈ alkyl and —(O(CH₂)₁₋₈)_{q}—OC₁₋₈ alkyl; and q is 1, 2, 3, 4, 5 or 6;
R² is unsubstituted C₁₋₆ alkyl or benzyl;
R³ is H or unsubstituted C₁₋₆ alkyl;
R⁴ is H or halogen;
R⁵ is H;
R⁶ is C₁₋₆ haloalkyl or bromine;
R⁷ is H;
R⁸ is H or halogen; and
n is 1 or 2;
or a single enantiomer thereof;
or a mixture of enantiomers thereof;
or a tautomer of the foregoing;
or pharmaceutically acceptable salt of the foregoing.

In some embodiments, there are provided compounds of Formula II, wherein R¹ is —COOH or —C(O)OR^{a}, wherein R^{a} is unsubstituted or substituted C₁₋₆ alkyl; wherein said alkyl substituent is selected from OH, halogen, —OC₁₋₈ alkyl and —(O(CH₂)₁₋₈)_{q}—OC₁₋₈ alkyl; and q is 1, 2, 3, 4, 5 or 6. In another embodiment, there are provided compound of Formula II, wherein R¹ is —COOH or —C(O)OR^{a}, and R^{a} is unsubstituted C₁₋₆ alkyl. In another embodiment, there are provided compound of Formula II, wherein R¹ is —COOH or —C(O)OR^{a}, and R^{a} is unsubstituted C₁₋₄ alkyl. In one embodiment, R¹ is —C(O)OR^{a}, and R^{a} is tert-butyl. In another embodiment, there are provided compounds of Formula II, wherein R¹ is —COOH.

In some embodiments, there are provided compounds of Formula II, wherein R¹ is —COOH, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole.

In some embodiments, there are provided compounds of Formula II, wherein R² is unsubstituted C₁₋₆ alkyl or benzyl. In some embodiments, R² is unsubstituted C₁₋₆ alkyl. In some embodiments, R² is unsubstituted C₁₋₄ alkyl, such as methyl, ethyl, n-propyl, isopropyl, n-butyl, tert-butyl, isobutyl or sec-butyl. In some embodiments, there are provided compounds of Formula II wherein R² is unsubstituted C₁₋₃ alkyl, such as methyl, ethyl, n-propyl or isopropyl. In another embodiment, R² is unsubstituted benzyl.

In some embodiments, there are provided compounds of Formula II, wherein R³ is H or unsubstituted C₁₋₃ alkyl. In one embodiment, embodiment, R³ is unsubstituted C₁₋₃ alkyl, such as methyl, ethyl, n-propyl or isopropyl. In yet another embodiment, R³ is methyl. In another embodiment, there are provided compounds of Formula II, wherein R³ is H.

In some embodiments, there are provided compounds of Formula II, wherein n is 1. In another embodiment, n is 2.

In some embodiments, there are provided compounds of Formula II, wherein R⁴ is H or F; and R⁸ is H or F. In another embodiment, there are provided compounds wherein R⁴ is H and R⁸ is F. In a further embodiment, there are provided compounds of Formula II, wherein at least one of R⁴ and R⁸ is H. In another embodiment, each of R⁴ and R⁸ is H.

In some embodiments, there are provided compounds of Formula II, wherein R⁶ is C₁₋₆ fluoroalkyl or bromine. In another embodiment, R⁶ is C₁₋₆ perfluoroalkyl or bromine. In another embodiment, R⁶ is C₁₋₃ perfluoroalkyl or bromine. In yet another embodiment, there are provided compounds of Formula II, wherein R⁶ is —CF₃ or bromine. In another embodiment, R⁶ is —CF₃. In another embodiment, R⁶ is —Br.

In another embodiment, there are provided compounds of Formula II, wherein:
R¹ is —COOH, —C(O)OR^{a}, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
   wherein R^{a} is optionally substituted C₁₋₆ alkyl, wherein said alkyl substituent is selected from OH, halogen, —OC₁₋₈ alkyl and —(O(CH₂)₁₋₈)_{q}—OC₁₋₈ alkyl; and q is 1, 2, 3, 4, 5 or 6;
R² is unsubstituted C₁₋₆ alkyl or benzyl;
R³ is H or unsubstituted C₁₋₆ alkyl;
R⁴ is H or F;
R⁵ is H;
R⁶ is C₁₋₆ fluoroalkyl or bromine;
R⁷ is H;
R⁸ is H or F; and
n is 1 or 2;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole; wherein R^{a} is unsubstituted C₁₋₆ alkyl;
R² is unsubstituted C₁₋₆ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole; wherein R^{a} is unsubstituted C₁₋₆ alkyl;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, thiophene, pyrazole and pyrrole; wherein R^{a} is unsubstituted C₁₋₆ alkyl;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole; wherein R^{a} is unsubstituted C₁₋₆ alkyl;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 2;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₆ alkyl or benzyl;
R³ is H or unsubstituted C₁₋₆ alkyl;
R⁴ is H or F;
R⁵ is H;
R⁶ is C₁₋₆ fluoroalkyl or bromine;
R⁷ is H;
R⁸ is H or F; and
n is 1 or 2;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₆ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a tautomer thereof;
or pharmaceutically acceptable salt of any one of the foregoing.

In another embodiment, there are provided compounds of Formula II, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, thiophene, pyrazole and pyrrole;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 2;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH or —C(O)OR^{a}, wherein R^{a} is unsubstituted C₁₋₆ alkyl;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1 or 2;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH or —C(O)OR^{a}, wherein R^{a} is unsubstituted C₁₋₆ alkyl;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH or —C(O)OR^{a}, wherein R^{a} is unsubstituted C₁₋₆ alkyl;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 2;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1 or 2;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 2;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH;
R² is unsubstituted C₁₋₃ alkyl or benzyl;
R³ is H;
R⁴ is H;
R⁵ is H;
R⁶ is —CF₃ or bromine;
R⁷ is H;
R⁸ is H; and
n is 1;
or a single enantiomer thereof;
or a mixture of enantiomers thereof;
or a tautomer of the foregoing;
or pharmaceutically acceptable salt of the foregoing.

In another embodiment, there are provided compounds of Formula II, wherein:
R¹ is —COOH;
R² is unsubstituted C₁₋₃ alkyl or benzyl;
R³ is H;
R⁴ is H;
R⁵ is H;
R⁶ is —CF₃ or bromine;
R⁷ is H;
R⁸ is H; and
n is 2;
or a single enantiomer thereof;
or a mixture of enantiomers thereof;
or a tautomer of the foregoing;
or pharmaceutically acceptable salt of the foregoing.

In some embodiments, provided herein are compounds of Formula IIa:

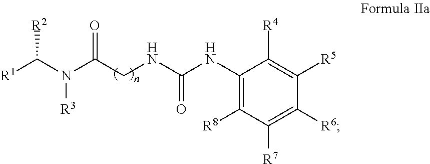

wherein the compound has the specific stereochemistry shown at the carbon bearing R²; and wherein:
R¹ is —COOH, —C(O)OR^{a}, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
   wherein R^{a} is optionally substituted C₁₋₆ alkyl, wherein said optional alkyl substituent is selected from OH, halogen, —OC₁₋₈ alkyl and —(O(CH₂)₁₋₈)_{q}—OC₁₋₈ alkyl; and q is 1, 2, 3, 4, 5 or 6;
R² is unsubstituted C₁₋₆ alkyl or benzyl;
R³ is H or unsubstituted C₁₋₆ alkyl;
R⁴ is H or halogen;
R⁵ is H;
R⁶ is C₁₋₆ haloalkyl or bromine;
R⁷ is H;
R⁸ is H or halogen; and
n is 1 or 2;
or a tautomer thereof;
or pharmaceutically acceptable salt of the foregoing.

In some embodiments, there are provided compounds of Formula IIa, wherein R¹ is —COOH or —C(O)OR^{a}, wherein R^{a} is unsubstituted or substituted C₁₋₆ alkyl; wherein said alkyl substituent is selected from OH, halogen, —OC₁₋₈ alkyl and —(O(CH₂)₁₋₈)_{q}—OC₁₋₈ alkyl; and q is 1, 2, 3, 4, 5 or 6. In another embodiment, there are provided compound of Formula IIa, wherein R¹ is —COOH or —C(O)OR^{a}, and R^{a} is unsubstituted C₁₋₆ alkyl. In another embodiment, there are provided compound of Formula IIa, wherein R¹ is —COOH or —C(O)OR^{a}, and R^{a} is unsubstituted C₁₋₄ alkyl. In one embodiment, R¹ is —C(O)OR^{a}, and R^{a} is tert-butyl. In another embodiment, there are provided compounds of Formula IIa, wherein R¹ is —COOH.

In some embodiments, there are provided compounds of Formula IIa, wherein R¹ is —COOH, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole.

In some embodiments, there are provided compounds of Formula IIa, wherein R² is unsubstituted C₁₋₆ alkyl or benzyl. In some embodiments, R² is unsubstituted C₁₋₆ alkyl. In some embodiments, R² is unsubstituted C₁₋₄ alkyl, such as methyl, ethyl, n-propyl, isopropyl, n-butyl, tert-butyl, isobutyl or sec-butyl. In some embodiments, there are provided compounds of Formula IIa wherein R² is unsubstituted C₁₋₃ alkyl, such as methyl, ethyl, n-propyl or isopropyl. In another embodiment, R² is unsubstituted benzyl.

In some embodiments, there are provided compounds of Formula IIa, wherein R³ is H or unsubstituted C₁₋₃ alkyl. In one embodiment, embodiment, R³ is unsubstituted C₁₋₃ alkyl, such as methyl, ethyl, n-propyl or isopropyl. In yet another embodiment, R³ is methyl. In another embodiment, there are provided compounds of Formula IIa, wherein R³ is H.

In some embodiments, there are provided compounds of Formula IIa, wherein n is 1. In another embodiment, n is 2.

In some embodiments, there are provided compounds of Formula IIa, wherein R⁴ is H or F; and R⁸ is H or F. In another embodiment, there are provided compounds wherein R⁴ is H and R⁸ is F. In a further embodiment, there are provided compounds of Formula IIa, wherein at least one of R⁴ and R⁸ is H. In another embodiment, each of R⁴ and R⁸ is H.

In some embodiments, there are provided compounds of Formula IIa, wherein R⁶ is C₁₋₆ fluoroalkyl or bromine. In another embodiment, R⁶ is C₁₋₆ perfluoroalkyl or bromine. In another embodiment, R⁶ is C₁₋₃ perfluoroalkyl or bromine. In yet another embodiment, there are provided compounds of Formula IIa, wherein R⁶ is —CF₃ or bromine. In another embodiment, R⁶ is —CF₃. In another embodiment, R⁶ is —Br.

In another embodiment, there are provided compounds of Formula IIa, wherein:
R¹ is —COOH, —C(O)OR^{a}, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
   wherein R^{a} is optionally substituted C₁₋₆ alkyl, wherein said alkyl substituent is selected from OH, halogen, —OC₁₋₈ alkyl and —(O(CH₂)₁₋₈)_{q}—OC₁₋₈ alkyl; and q is 1, 2, 3, 4, 5 or 6;
R² is unsubstituted C₁₋₆ alkyl or benzyl;
R³ is H or unsubstituted C₁₋₆ alkyl;
R⁴ is H or F;
R⁵ is H;
R⁶ is C₁₋₆ fluoroalkyl or bromine;
R⁷ is H;
R⁸ is H or F; and
n is 1 or 2;
or a tautomer thereof;
or pharmaceutically acceptable salt of any one of the foregoing.

In another embodiment, there are provided compounds of Formula IIa, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, thiophene, pyrazole and pyrrole; wherein R^{a} is unsubstituted C₁₋₆ alkyl;
R² is unsubstituted C₁₋₆ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a tautomer thereof;
or pharmaceutically acceptable salt of any one of the foregoing.

In another embodiment, there are provided compounds of Formula IIa, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole; wherein R^{a} is unsubstituted C₁₋₆ alkyl;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a tautomer thereof;
or pharmaceutically acceptable salt of any one of the foregoing.

In another embodiment, there are provided compounds of Formula IIa, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole; wherein R^{a} is unsubstituted C₁₋₆ alkyl;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1;
or a tautomer thereof;
or pharmaceutically acceptable salt of any one of the foregoing.

In another embodiment, there are provided compounds of Formula IIa, wherein:
R¹ is —COOH, —C(O)OR^{a}, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole; wherein R^{a} is unsubstituted C₁₋₆ alkyl;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 2;
or a tautomer thereof;
or pharmaceutically acceptable salt of any one of the foregoing.

In another embodiment, there are provided compounds of Formula IIa, wherein:
R¹ is —COOH, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₆ alkyl or benzyl;
R³ is H or unsubstituted C₁₋₆ alkyl;
R⁴ is H or F;
R⁵ is H;
R⁶ is C₁₋₆ fluoroalkyl or bromine;
R⁷ is H;
R⁸ is H or F; and
n is 1 or 2;
or a tautomer thereof;
or pharmaceutically acceptable salt of the foregoing.

In another embodiment, there are provided compounds of Formula IIa, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₆ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a tautomer thereof;
or pharmaceutically acceptable salt of the foregoing.

In another embodiment, there are provided compounds of Formula IIa, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1 or 2;
or a tautomer thereof;
or pharmaceutically acceptable salt of the foregoing.

In another embodiment, there are provided compounds of Formula IIa, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, thiophene, pyrazole and pyrrole;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 1;
or a tautomer thereof;
or pharmaceutically acceptable salt of the foregoing.

In another embodiment, there are provided compounds of Formula IIa, wherein:
R¹ is —COOH, sulfonate, sulfonic acid, phosphonate, phosphonic acid, phosphoric acid, or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
R² is unsubstituted C₁₋₄ alkyl or benzyl;
R³ is H or unsubstituted C₁₋₃ alkyl;
R⁴ is H;
R⁵ is H;
R⁶ is C₁₋₆ fluoroalkyl or bromine;
R⁷ is H;
R⁸ is H; and
n is 2;
or a tautomer thereof;
or pharmaceutically acceptable salt of the foregoing.

In another embodiment, there are provided compounds of Formula IIa, wherein:
R¹ is —COOH or —C(O)OR^{a}, wherein R^{a} is unsubstituted C₁₋₆ alkyl;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1 or 2;
or a tautomer thereof;
or pharmaceutically acceptable salt of any one of the foregoing.

In another embodiment, there are provided compounds of Formula IIa, wherein:
R¹ is —COOH or —C(O)OR^{a}, wherein R^{a} is unsubstituted C₁₋₆ alkyl;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1;
or a tautomer thereof;
or pharmaceutically acceptable salt of any one of the foregoing.

In another embodiment, there are provided compounds of Formula IIa, wherein:
R¹ is —COOH or —C(O)OR^{a}, wherein R^{a} is unsubstituted C₁₋₆ alkyl;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 2;
or a tautomer thereof;
or pharmaceutically acceptable salt of any one of the foregoing.

In another embodiment, there are provided compounds of Formula IIa, wherein:
R¹ is —COOH;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1 or 2;
or a tautomer thereof;
or pharmaceutically acceptable salt of the foregoing.

In another embodiment, there are provided compounds of Formula IIa, wherein:
R¹ is —COOH;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1;
or a tautomer thereof;
or pharmaceutically acceptable salt of the foregoing.

In another embodiment, there are provided compounds of Formula IIa, wherein:
R¹ is —COOH;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 2;
or a tautomer thereof;
or pharmaceutically acceptable salt of the foregoing.

In another embodiment, there are provided compounds of Formula IIa, wherein:
R¹ is —COOH;
R² is unsubstituted C₁₋₃ alkyl or benzyl;
R³ is H;
R⁴ is H;
R⁵ is H;
R⁶ is —CF₃ or bromine;
R⁷ is H;
R⁸ is H; and
n is 1;
or a tautomer thereof;
or pharmaceutically acceptable salt of any one of the foregoing.

In another embodiment, there are provided compounds of Formula IIa, wherein:
R¹ is —COOH;
R² is unsubstituted C₁₋₃ alkyl or benzyl;
R³ is H;
R⁴ is H;
R⁵ is H;
R⁶ is —CF₃ or bromine;
R⁷ is H;
R⁸ is H; and
n is 2;
or a tautomer thereof;
or pharmaceutically acceptable salt of any one of the foregoing.

In some embodiments, the invention provides for a compound selected from:
(S)-2-(2-(3-(4-bromophenyl)ureido)acetamido)-4-methylpentanoic acid;
(S)-4-methyl-2-(2-(3-(4-(trifluoromethyl)phenyl)ureido)acetamido)pentanoic acid;
(S)-2-(2-(3-(4-bromophenyl)ureido)acetamido)-3-methylbutanoic acid;
(S)-2-(2-(3-(4-bromophenyl)ureido)acetamido)-3-phenylpropanoic acid;
(S)-2-(3-(3-(4-bromophenyl)ureido)propanamido)-4-methylpentanoic acid;
(S)-2-(2-(3-(4-bromophenyl)ureido)acetamido)pentanoic acid;
(S)-2-(2-(3-(4-(trifluoromethyl)phenyl)ureido)acetamido)pentanoic acid;
(S)-tert-butyl 2-(2-(3-(4-bromophenyl)ureido)acetamido)pentanoate; and
(S)-tert-butyl 2-(2-(3-(4-(trifluoromethyl)phenyl)ureido)acetamido) pentanoate;
and pharmaceutically acceptable salts thereof.

In some embodiments, the invention provides for a compound selected from:

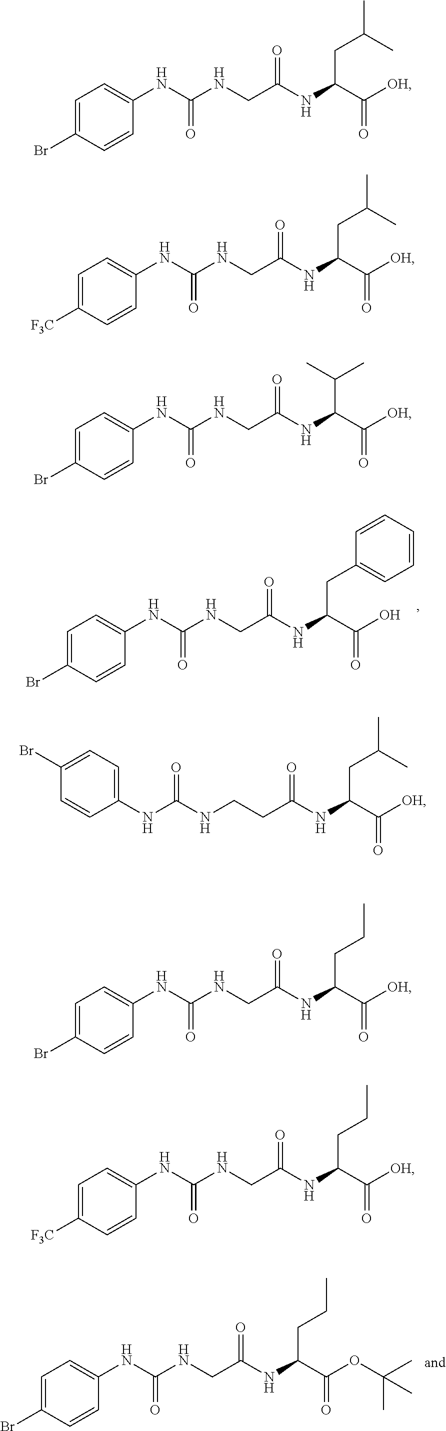

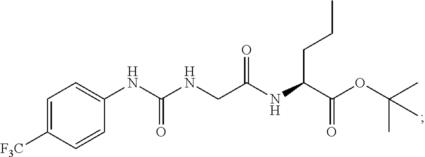 [SMILES: CCC[C@H](NC(=O)CNC(=O)Nc1ccc(C(F)(F)F)cc1)C(=O)OC(C)(C)C]

and pharmaceutically acceptable salts thereof.

In some embodiments, the invention provides for a compound selected from:

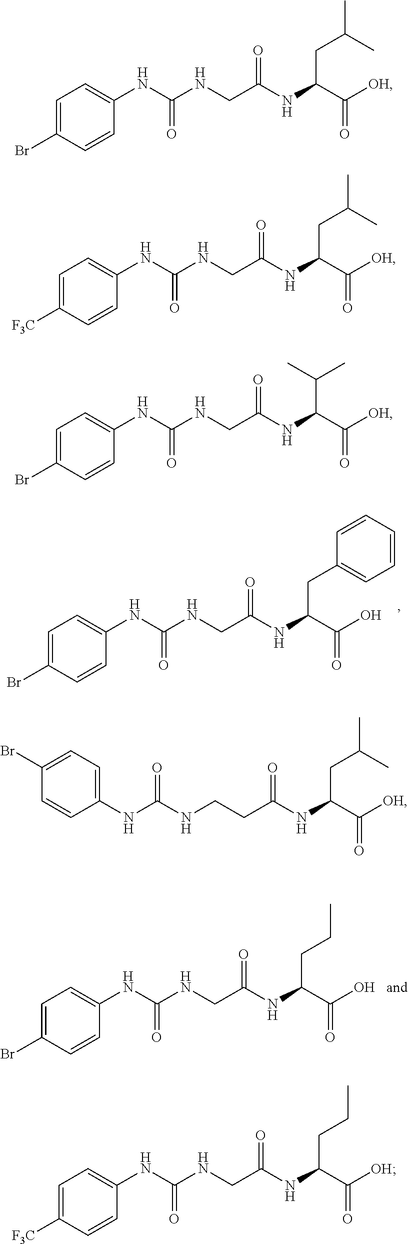

and pharmaceutically acceptable salts thereof.

Some compounds of the invention may form salts with acids or bases, including pharmaceutically acceptable acids or bases. Such pharmaceutically acceptable salts of the compounds described herein are within the scope of the invention.

The acid addition salt form of a compound of Formula I, Ia, II or IIa that occurs in its free form as a base can be obtained by treating the free base with an appropriate acid such as an inorganic acid, for example, hydrochloric acid, hydrobromic acid, sulfuric acid, phosphoric acid, nitric acid and the like; or an organic acid such as for example, acetic acid, hydroxyacetic acid, propanoic acid, lactic acid, pyruvic acid, malonic acid, fumaric acid, maleic acid, oxalic acid, tartaric acid, succinic acid, malic acid, ascorbic acid, benzoic acid, tannic acid, pamoic acid, citric acid, methylsulfonic acid, ethanesulfonic acid, benzenesulfonic acid, formic acid and the like. The base addition salt form of a compound of Formula I, Ia, II or IIa that occurs in its acid form can be obtained by treating the acid with an appropriate base such as an inorganic base, for example, sodium hydroxide, magnesium hydroxide, potassium hydroxide, calcium hydroxide, ammonia and the like; or an organic base such as for example, L-arginine, ethanolamine, betaine, benzathine, morpholine and the like. (See Handbook of Pharmaceutical Salts, P. Heinrich Stahl & Camille G. Wermuth (Eds), Verlag Helvetica Chimica Acta, Zirich, 2002, 329-345.) Some of the compounds of Formula I, Ia, II or IIa and some of their intermediates may contain one or more asymmetric centers in their structure; each asymmetric center may be present in an R or S configuration, said R and S notation corresponding to the rules described in Pure and Applied Chemistry (1976), 45, 11-13. As such, the compounds may exist in enantiomeric as well as in diastereomeric forms. Unless it is specifically noted otherwise, the scope of the present invention includes all enantiomers, diastereomers and mixtures thereof, including racemic mixtures.

As will be evident to those skilled in the art, individual diastereoisomeric forms can be obtained by separation of mixtures thereof in a conventional manner. For example, chromatographic separation may be employed; chiral chromatography may be performed to separate individual enantiomers.

The present invention includes all pharmaceutically acceptable isotopically enriched compounds. Any compound of the invention may contain one or more isotopic atoms enriched or different than the natural ratio, such as deuterium ²H (or D) in place of hydrogen ¹H (or H), or use of ¹³C enriched material in place of ¹²C and the like. Similar substitutions can be employed for N, O, S and P. The use of isotopes may assist in analytical as well as therapeutic aspects of the invention. For example, use of deuterium may increase the in vivo half-life by altering the metabolism (rate) of the compounds of the invention. These compounds can be prepared in accord with the preparations described by use of isotopically enriched reagents.

In an embodiment of the invention, there are provided pharmaceutical compositions including a therapeutically effective amount of at least one compound of the invention in a pharmaceutically acceptable carrier.

The compounds of the invention and the pharmaceutical compositions comprising at least one compound of the invention are indicated for use in treating or preventing conditions in which there is likely to be a component involving the FPR, such as FPR1 and/or FPR2.

In a further embodiment of the invention, there are provided methods for treating disorders associated with FPR modulation, such as FPR1 and/or FPR2 agonism, or selective agonism of FPR1 relative to FPR2. Such methods can be performed, for example, by administering to a subject in need thereof a pharmaceutical composition comprising a therapeutically effective amount of at least one compound of the invention.

More specifically, the present invention provides for:

use of a compound of the invention in the manufacture of a medicament for the treatment of a mammalian subject, including a human subject, having one or more diseases or conditions that are alleviated by FPR modulation (such as FPR1 and/or FPR2 agonism, or selective agonism of FPR1 relative to FPR2); and/or

a method of treating a mammalian subject, including a human subject, having one or more diseases or conditions that are alleviated by FPR modulation (such as FPR1 and/or FPR2 agonism, or selective agonism of FPR1 relative to FPR2);

wherein the disease or condition is an ocular disease or condition, including but not limited to: ocular inflammation, age-related macular degeneration, wet macular degeneration, dry macular degeneration, uveitis, dry eye, keratitis, allergic eye disease and conditions affecting the posterior part of the eye, such as maculopathies and retinal degeneration including non-exudative age related macular degeneration, exudative age related macular degeneration, choroidal neovascularization, diabetic retinopathy (proliferative), retinopathy of prematurity, acute macular neuroretinopathy, central serous chorioretinopathy, cystoid macular edema, and diabetic macular edema; infectious keratitis, herpetic keratitis, corneal angiogenesis, lymphangiogenesis, uveitis, retinitis, choroiditis, such as acute multifocal placoid pigment epitheliopathy, Behcet's disease, birdshot retinochoroidopathy, infectious (syphilis, lyme, tuberculosis, toxoplasmosis), intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi- and Harada syndrome; vascular diseases/exudative diseases such as retinal arterial occlusive disease, central retinal vein occlusion, cystoids macular edema, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angiitis, sickle cell retinopathy and other hemoglobinopathies, angioid streaks, familial exudative vitreoretinopathy, and Eales disease; traumatic/surgical conditions such as sympathetic ophthalmia, uveitic retinal disease, retinal detachment, trauma, conditions caused by laser, conditions caused by photodynamic therapy, photocoagulation, hypoperfusion during surgery, radiation retinopathy, bone marrow transplant retinopathy, corneal wound healing, post-surgical corneal wound healing and/or inflammation, and post-cataract surgical inflammation; proliferative disorders such as proliferative vitreal retinopathy and epiretinal membranes, and proliferative diabetic retinopathy; infectious disorders such as ocular histoplasmosis, ocular toxocariasis, presumed ocular histoplasmosis syndrome (POHS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; and miscellaneous other diseases affecting the posterior part of the eye such as punctate inner choroidopathy, acute posterior multifocal placoid pigment epitheliopathy, myopic retinal degeneration, and acute retinal pigment epitheliitis, blepharitis, meibomian gland dysfunction (MDG), glaucoma, branch vein occlusion, Best's vitelliform macular degeneration, retinitis pigmentosa, proliferative vitreoretinopathy (PVR), and any other degenerative disease of either the photoreceptors or the retinal pigment epithelium (Perretti, Mauro et al. Pharmacology & Therapeutics 127 (2010) 175-188).

In other embodiments, the present invention provides for:

use of a compound of the invention in the manufacture of a medicament for the treatment of a mammalian subject, including a human subject, having one or more diseases or conditions that are alleviated by FPR modulation (such as FPR1 and/or FPR2 agonism, or selective agonism of FPR1 relative to FPR2); and/or

a method of treating a mammalian subject, including a human subject, having one or more diseases or conditions that are alleviated by FPR modulation (such as FPR1 and/or FPR2 agonism, or selective agonism of FPR1 relative to FPR2);

wherein the disease or condition is a dermal disease or condition, including, but not limited to: dermal inflammation, dermal wound healing, hypertrophic scars, keloids, burns, sunburn, rosacea, erythema of the skin, atopic dermatitis, acne, psoriasis, seborrheic dermatitis, actinic keratoses, basal cell carcinoma, squamous cell carcinoma, melanoma, viral warts, photoaging, photodamage, melasma, post-inflammatory hyperpigmentation, disorders of pigmentation, alopecia, scarring and non-scarring forms.

In yet other embodiments, the present invention provides for:

use of a compound of the invention in the manufacture of a medicament for the treatment of a mammalian subject, including a human subject, having one or more diseases or conditions that are alleviated by FPR modulation (such as FPR1 and/or FPR2 agonism, or selective agonism of FPR1 relative to FPR2); and/or

a method of treating a mammalian subject, including a human subject, having one or more diseases or conditions that are alleviated by FPR modulation (such as FPR1 and/or FPR2 agonism, or selective agonism of FPR1 relative to FPR2);

wherein the disease or condition is selected from: a systemic inflammatory disease or condition, stroke, coronary artery disease, a cardiovascular disorder, coronary artery disease, angina pectoris; or

an obstructive airway disease; or

a neurological disorder, a central nervous system disorder, Alzheimer's disease, neuroinflammation or pain; or

an HIV-mediated retroviral infection; or

an immunological disorder, arthritis, rheumatoid arthritis, systemic lupus erythematosus, multiple sclerosis; or

sepsis; or

inflammatory bowel disease or ulcerative colitis; or

asthma or an allergic disorder; or

cachexia.

In a further embodiment of the invention, the method of treating a disease or condition alleviated by FPR modulation (such as FPR1 and/or FPR2 agonism, or selective agonism of FPR1 relative to FPR2), comprises administering to the subject in need of the treatment a therapeutically effective amount of at least one compound of the invention, or an enantiomer, diastereomer or tautomer thereof, or pharmaceutically acceptable salt of any one of the foregoing.

In one embodiment, the invention provides for a method of treating a disease or condition in a subject in need of such treatment, the method comprising administering a therapeutically effective amount of a compound of Formula I, Ia, II or IIa, to the subject, thereby treating the disease or condition. In one embodiment, the method comprises administering a compound of Formula I. In another embodiment, the method comprises administering a compound of Formula Ia. In another embodiment, the method comprises administering a compound of Formula II. In another embodiment, the method comprises administering a compound of Formula IIa.

In another embodiment, the invention provides for a method of treating a disease or condition in a subject in need of such treatment, the method comprising administering a pharmaceutical composition comprising a therapeutically effective amount of a compound of Formula I, Ia, II or IIa to the subject, thereby treating the disease or condition. In one embodiment, the method comprises administering a pharmaceutical composition comprising a therapeutically effective amount of a compound of Formula I. In one embodiment, the method comprises administering a pharmaceutical composition comprising a therapeutically effective amount of a compound of Formula Ia. In one embodiment, the method comprises administering a pharmaceutical composition comprising a therapeutically effective amount of a compound of Formula II. In one embodiment, the method comprises administering a pharmaceutical composition comprising a therapeutically effective amount of a compound of Formula IIa.

In another embodiment, there is provided a method of treating the disease or condition associated with FPR modulation (such as FPR1 and/or FPR2 agonism, or selective agonism of FPR1 relative to FPR2) in a subject in need of such treatment, wherein the disease or condition is an ocular disease or condition; the method comprising administering a pharmaceutical composition comprising a therapeutically effective amount of a compound of Formula I, Ia, II or IIa to the subject, thereby treating the disease or condition. In a further embodiment, the ocular disease or condition selected from: ocular inflammation, age-related macular degeneration, wet macular degeneration, dry macular degeneration, uveitis, dry eye, keratitis, allergic eye disease and conditions affecting the posterior part of the eye, such as maculopathies and retinal degeneration including non-exudative age related macular degeneration, exudative age related macular degeneration, choroidal neovascularization, diabetic retinopathy (proliferative), retinopathy of prematurity, acute macular neuroretinopathy, central serous chorioretinopathy, cystoid macular edema, and diabetic macular edema; infectious keratitis, herpetic keratitis, corneal angiogenesis, lymphangiogenesis, retinitis, and choroiditis such as acute multifocal placoid pigment epitheliopathy, Behcet's disease, birdshot retinochoroidopathy, infectious (syphilis, lyme, tuberculosis, toxoplasmosis), intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi- and Harada syndrome; vascular diseases/exudative diseases such as retinal arterial occlusive disease, central retinal vein occlusion, cystoids macular edema, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angiitis, sickle cell retinopathy and other hemoglobinopathies, angioid streaks, familial exudative vitreoretinopathy, and Eales disease; traumatic/surgical conditions such as sympathetic ophthalmia, uveitic retinal disease, retinal detachment, trauma, conditions caused by laser, conditions caused by photodynamic therapy, photocoagulation, hypoperfusion during surgery, radiation retinopathy, bone marrow transplant retinopathy, post-surgical corneal wound healing or inflammation, and post-cataract surgical inflammation; proliferative disorders such as proliferative vitreal retinopathy and epiretinal membranes, and proliferative diabetic retinopathy; infectious disorders such as ocular histoplasmosis, ocular toxocariasis, presumed ocular histoplasmosis syndrome (POHS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; and miscellaneous other diseases affecting the posterior part of the eye such as punctate inner choroidopathy, acute posterior multifocal placoid pigment epitheliopathy, myopic retinal degeneration, and acute retinal pigment epitheliitis, post-surgical corneal inflammation, blepharitis, meibomian gland dysfunction (MGD), corneal wound healing, glaucoma, branch vein occlusion, Best's vitelliform macular degeneration, retinitis pigmentosa, proliferative vitreoretinopathy (PVR), and any other degenerative disease of either the photoreceptors or the retinal pigment epithelium. In a further embodiment, the ocular inflammatory disease or condition is selected from: dry eye, a post-surgical corneal wound, post-surgical corneal inflammation, and post-cataract surgical inflammation. In a further embodiment, the subject is a human.

In another embodiment, there is provided a method of treating the disease or condition associated with FPR modulation (such as FPR1 and/or FPR2 agonism, or selective agonism of FPR1 relative to FPR2) in a subject in need of such treatment, wherein the disease or condition is a dermal disease or condition; the method comprising administering a pharmaceutical composition comprising a therapeutically effective amount of a compound of Formula I, Ia, II or IIa to the subject, thereby treating the disease or condition. In a further embodiment, the dermal disease or condition is selected from: dermal inflammation, a dermal wound, hypertrophic scars, keloids, burns, sunburn, rosacea, erythema of the skin, atopic dermatitis, acne, psoriasis, seborrheic dermatitis, actinic keratoses, basal cell carcinoma, squamous cell carcinoma, melanoma, viral warts, photoaging, photodamage, melasma, post-inflammatory hyperpigmentation, disorders of pigmentation, alopecia, scarring and non-scarring forms. In a further embodiment, the dermal disease or condition is psoriasis or rosacea. In a further embodiment, the subject is a human.

In another embodiment, there is provided the method of treating the disease or condition associated with FPR modulation (such as FPR1 and/or FPR2 agonism, or FPR1 agonism, or selective agonism of FPR1 relative to FPR2) in a subject in need of such treatment, wherein the disease or condition is a systemic inflammatory condition, stroke, coronary artery disease, a cardiovascular disorder, coronary artery disease or angina pectoris; or an obstructive airway disease; or a neurological disorder, Alzheimer's disease, neuroinflammation or pain; or an HIV-mediated retroviral infection; or an immunological disorder, arthritis, rheumatoid arthritis, systemic lupus erythematosus, multiple sclerosis; or sepsis; or inflammatory bowel disease or ulcerative colitis; or asthma or an allergic disorder; or cachexia; the method comprising administering a pharmaceutical composition comprising a therapeutically effective amount of a compound of Formula I, Ia, II or IIa to the subject, thereby treating the disease or condition. In a further embodiment, the disease or condition is rheumatoid arthritis. In one embodiment, the disease or condition is multiple sclerosis. In another embodiment, the disease or condition is inflammatory bowel disease. In one embodiment, the disease or condition is ulcerative colitis. In a further embodiment, the condition is pain. In a further embodiment, the subject is a human.

The actual amount of the compound to be administered in any given case will be determined by a physician taking into account the relevant circumstances, such as the severity of the condition, the age and weight of the subject/patient, the patient's general physical condition, the cause of the condition, and the route of administration.

The subject will be administered the compound orally in any acceptable form, such as a tablet, liquid, capsule, powder and the like, or other routes may be desirable or necessary, particularly if the patient suffers from nausea. Such other routes may include, without exception, transdermal, parenteral, subcutaneous, intranasal, via an implant stent, intrathecal, intravitreal, topical to the eye, back of the eye, intramuscular, intravenous, and intrarectal modes of delivery. Additionally, the formulations may be designed to delay release of the active compound over a given period of time, or to carefully control the amount of drug released at a given time during the course of therapy.

In another embodiment of the invention, there are provided pharmaceutical compositions including at least one compound of the invention in a pharmaceutically acceptable carrier thereof. The phrase “pharmaceutically acceptable” means the carrier, diluent or excipient must be compatible with the other ingredients of the formulation and not deleterious to the recipient thereof.

Pharmaceutical compositions of the present invention can be used in the form of a solid, a solution, an emulsion, a dispersion, a patch, a micelle, a liposome, and the like, wherein the resulting composition contains one or more compounds of the present invention, as an active ingredient, in admixture with an organic or inorganic carrier or excipient suitable for enteral or parenteral applications. Invention compounds may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition, auxiliary, stabilizing, thickening and coloring agents and perfumes may be used. Invention compounds are included in the pharmaceutical composition in an amount sufficient to produce the desired effect upon the process or disease condition.

Pharmaceutical compositions containing invention compounds may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing invention compounds in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

In some cases, formulations for oral use may be in the form of hard gelatin capsules wherein the invention compounds are mixed with an inert solid diluent, for example, calcium carbonate, calcium phosphate or kaolin. They may also be in the form of soft gelatin capsules wherein the invention compounds are mixed with water or an oil medium, for example, peanut oil, liquid paraffin or olive oil.

The pharmaceutical compositions may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose, any bland fixed oil may be employed, including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

Pharmaceutical compositions containing invention compounds may be in a form suitable for topical use, for example, as oily suspensions, as solutions or suspensions in aqueous liquids or nonaqueous liquids, or as oil-in-water or water-in-oil liquid emulsions. Pharmaceutical compositions may be prepared by combining a therapeutically effective amount of at least one compound according to the present invention, or a pharmaceutically acceptable salt thereof, as an active ingredient with conventional ophthalmically acceptable pharmaceutical excipients and by preparation of unit dosage suitable for topical ocular use. The therapeutically efficient amount typically is between about 0.001 and about 5% (w/v), preferably about 0.001 to about 2.0% (w/v) in liquid formulations.

For ophthalmic application, preferably solutions are prepared using a physiological saline solution as a major vehicle. The pH of such ophthalmic solutions should preferably be maintained between 4.5 and 8.0 with an appropriate buffer system, a neutral pH being preferred but not essential. The formulations may also contain conventional pharmaceutically acceptable preservatives, stabilizers and surfactants. Preferred preservatives that may be used in the pharmaceutical compositions of the present invention include, but are not limited to, benzalkonium chloride, chlorobutanol, thimerosal, phenylmercuric acetate and phenylmercuric nitrate. A preferred surfactant is, for example, Tween 80. Likewise, various preferred vehicles may be used in the ophthalmic preparations of the present invention. These vehicles include, but are not limited to, polyvinyl alcohol, povidone, hydroxypropyl methyl cellulose, poloxamers, carboxymethyl cellulose, hydroxyethyl cellulose cyclodextrin and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar manner an ophthalmically acceptable antioxidant for use in the present invention includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. The preferred chelating agent is edentate disodium, although other chelating agents may also be used in place of or in conjunction with it.

The ingredients are usually used in the following amounts:

| Ingredient | Amount (% w/v) |
|---|---|
| active ingredient | about 0.001-5 |
| preservative | 0-0.10 |
| vehicle | 0-40 |
| tonicity adjustor | 0-10 |
| buffer | 0.01-10 |
| pH adjustor | q.s. pH 4.5-7.8 |
| antioxidant | as needed |
| surfactant | as needed |
| purified water | to make 100% |

The actual dose of the active compounds of the present invention depends on the specific compound, and on the condition to be treated; the selection of the appropriate dose is well within the knowledge of the skilled artisan.

The ophthalmic formulations of the present invention are conveniently packaged in forms suitable for metered application, such as in containers equipped with a dropper, to facilitate application to the eye. Containers suitable for dropwise application are usually made of suitable inert, non-toxic plastic material, and generally contain between about 0.5 and about 15 ml solution. One package may contain one or more unit doses. Preservative-free solutions are often formulated in non-resalable containers containing up to about ten, preferably up to about five units doses, where a typical unit dose is from one to about 8 drops, preferably one to about 3 drops. The volume of one drop usually is about 20-35 microliters.

The compounds of the invention may also be administered in the form of suppositories for rectal administration of the drug. These compositions may be prepared by mixing the invention compounds with a suitable non-irritating excipient, such as cocoa butter, synthetic glyceride esters of polyethylene glycols, which are solid at ordinary temperatures, but liquefy and/or dissolve in the rectal cavity to release the drug.

Since individual subjects may present a wide variation in severity of symptoms and each drug has its unique therapeutic characteristics, the precise mode of administration and dosage employed for each subject is left to the discretion of the practitioner.

In one embodiment, the invention provides for a pharmaceutical composition comprising as active ingredient a therapeutically effective amount of a compound of Formula I, Ia, II or IIa, and a pharmaceutically acceptable carrier. In a further embodiment, there is provided a compound of Formula I, Ia, II or IIa, or pharmaceutical composition comprising a compound of Formula I, Ia, II or IIa, or any compound specifically set forth herein, for use in treating an inflammatory disease or condition in a subject in need of such treatment, wherein the disease or condition is an ocular inflammatory disease or condition, a dermal inflammatory disease or condition, a systemic inflammatory disease or condition, or an autoimmune disease or condition.

Nonlimiting embodiments of the invention are listed below:

(1) A compound of Formula I:

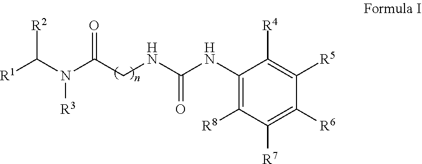

wherein:
R¹ is —COOH, —C(O)OR^{a}, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole; and R^{a} is optionally substituted C₁₋₆ alkyl; wherein said optional alkyl substituent is selected from OH, halogen, —OC₁₋₈alkyl and —(O(CH₂)₁₋₈)_{q}—OC₁₋₈ alkyl; and q is 1, 2, 3, 4, 5 or 6;
R² is optionally substituted C₁₋₆alkyl, wherein said optional alkyl substituent is selected from —OH, —SH, —OC₁₋₆ alkyl, —SC₁₋₆ alkyl, —COOH, —C(O)OC₁₋₆ alkyl, —C(O)NH₂, optionally substituted C₃₋₈cycloalkyl, optionally substituted C₃₋₈cycloalkenyl, optionally substituted C₆₋₁₀aryl, and optionally substituted heterocycle;
R³ is H or unsubstituted C₁₋₆ alkyl;
R⁴ is H, optionally substituted C₁₋₆ alkyl, C₁₋₆ haloalkyl, optionally substituted C₃₋₈ cycloalkyl, optionally substituted C₃₋₈ cycloalkenyl, optionally substituted C₆₋₁₀ aryl, optionally substituted heterocycle, halogen, —NR¹¹R¹², —S(O)ₘR⁹, —C(O)R¹⁰, —C(O)OC₁₋₆ alkyl, —C(O)SC₁₋₆ alkyl, —OR¹³ or —SR¹³;
R⁵ is H, optionally substituted C₁₋₆alkyl, C₁₋₆haloalkyl, halogen, —S(O)ₘR⁹ or —C(O)R¹⁰;
R⁶ is optionally substituted C₁₋₆alkyl, C₁₋₆haloalkyl, optionally substituted C₃₋₈ cycloalkyl, optionally substituted C₃₋₈cycloalkenyl, optionally substituted C₆₋₁₀ aryl, optionally substituted heterocycle, halogen, —S(O)ₘR⁹, —C(O)R¹⁰ or —OR¹¹;
R⁷ is H, optionally substituted C₁₋₆alkyl, C₁₋₆haloalkyl, halogen, —S(O)ₘR⁹ or —C(O)R¹⁰;
R⁸ is H, optionally substituted C₁₋₆ alkyl, C₁₋₆ haloalkyl, optionally substituted C₃₋₈ cycloalkyl, optionally substituted C₃₋₈ cycloalkenyl, optionally substituted C₆₋₁₀ aryl, optionally substituted heterocycle, halogen, —NR¹¹R¹², —S(O)ₘR⁹, —C(O)R¹⁰, —C(O)OC₁₋₆ alkyl, —C(O)SC₁₋₆ alkyl, —OR¹³ or —SR¹³;
each R⁹ is independently —OH, optionally substituted C₁₋₆alkyl or optionally substituted C₆₋₁₀aryl;
each R¹⁰ is independently —OH, optionally substituted C₁₋₆alkyl or optionally substituted C₆₋₁₀aryl;
each R¹¹ is independently H, optionally substituted C₁₋₈alkyl, optionally substituted C₃₋₈ cycloalkyl, optionally substituted C₃₋₈cycloalkenyl, optionally substituted C₆₋₁₀ aryl or optionally substituted heterocycle;
each R¹² is independently H, optionally substituted C₁₋₈alkyl, optionally substituted C₃₋₈ cycloalkyl, optionally substituted C₃₋₈cycloalkenyl, optionally substituted C₆₋₁₀aryl or optionally substituted heterocycle;
each R¹³ is independently H or optionally substituted C₁₋₈ alkyl;
each m is independently 1 or 2; and
n is 1, 2 or 3;
or a single enantiomer thereof;
or a mixture of enantiomers thereof;
or a tautomer of the foregoing;
or pharmaceutically acceptable salt of the foregoing;
provided that the compound is not:

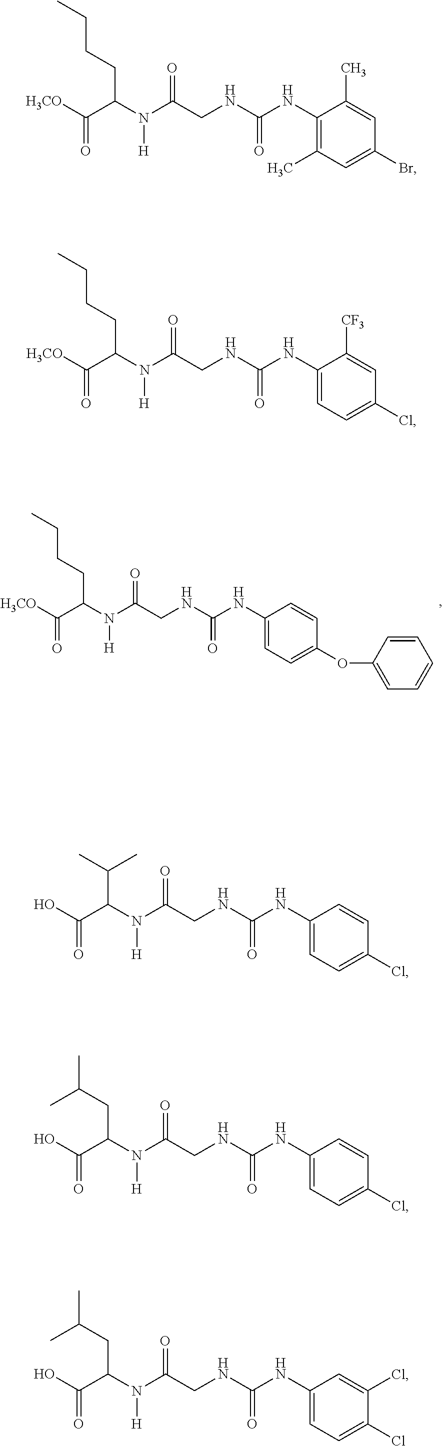 [SMILES: CCCCC(NC(=O)CNC(=O)Nc1c(C)cc(Br)cc1C)C(=O)OC ; CCCCC(NC(=O)CNC(=O)Nc1ccc(Cl)cc1C(F)(F)F)C(=O)OC ; CCCCC(NC(=O)CNC(=O)Nc1ccc(Oc2ccccc2)cc1)C(=O)OC ; CC(C)C(NC(=O)CNC(=O)Nc1ccc(Cl)cc1)C(=O)O ; CC(C)CC(NC(=O)CNC(=O)Nc1ccc(Cl)cc1)C(=O)O ; CC(C)CC(NC(=O)CNC(=O)Nc1ccc(Cl)c(Cl)c1)C(=O)O]

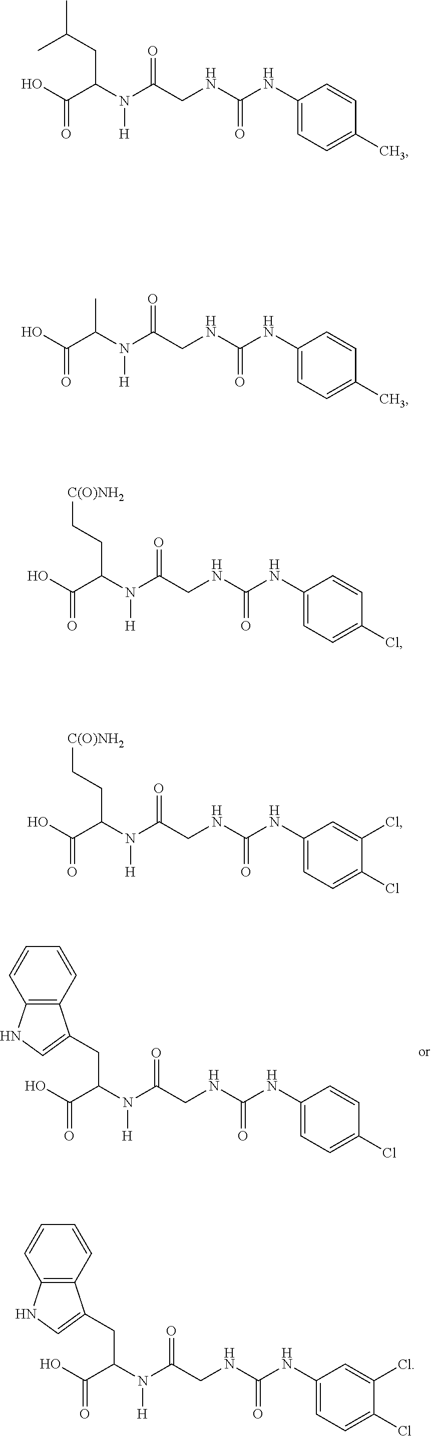

(2) The compound of embodiment (1), wherein R¹ is —COOH, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole.
(3) The compound of embodiment (1) or (2), wherein R¹ is —COOH.
(4) The compound of any one of embodiments (1) through (3), wherein R² is unsubstituted C₁₋₆alkyl or benzyl.
(5) The compound of any one of embodiments (1) through (4), wherein R³ is H or methyl.
(6) The compound of any one of embodiments (1) through (5), wherein n is 1 or 2.
(7) The compound of any one of embodiments (1) through (6), wherein R⁴ is H, F or C₁₋₆haloalkyl; R⁵ is H, F or C₁₋₆haloalkyl; R⁷ is H, F or C₁₋₆haloalkyl; and R⁸ is H, F or C₁₋₆ haloalkyl.
(8) The compound of embodiment (1) or (7), wherein each of R⁴, R⁵, R⁷ and R⁸ is H.
(9) The compound of any one of embodiments (1) through (8), wherein R⁶ is C₁₋₆haloalkyl or halogen.
(10) The compound of embodiment (1), provided that R⁶ is not chlorine, methyl or —OC₆₋₁₀aryl.
(11) The compound of embodiment (1), wherein:
R¹ is —COOH, —C(O)OR^{a}, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
   wherein R^{a} is optionally substituted C₁₋₆ alkyl, wherein said optional alkyl substituent is selected from OH, halogen, —OC₁₋₆ alkyl and —(O(CH₂)₁₋₈)_{q}—OC₁₋₈ alkyl; and q is 1, 2, 3, 4, 5 or 6;
R² is unsubstituted C₁₋₆ alkyl or benzyl;
R³ is H or unsubstituted C₁₋₆ alkyl;
R⁴ is H or halogen;
R⁵ is H;
R⁶ is C₁₋₆ haloalkyl or bromine;
R⁷ is H;
R⁸ is H or halogen; and
n is 1 or 2;
or a single enantiomer thereof;
or a mixture of enantiomers thereof;
or a tautomer of the foregoing;
or pharmaceutically acceptable salt of the foregoing.
   (12) The compound of embodiment (11), wherein R¹ is —COOH, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole; preferably, R¹ is —COOH.
   (13) The compound of embodiment (11) or (12), wherein R⁴ and R⁸ is H.
   (14) The compound of embodiment (11), wherein:
R¹ is —COOH or —C(O)OR^{a}, wherein R^{a} is unsubstituted C₁₋₆ alkyl;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1 or 2;
or a tautomer thereof;
or pharmaceutically acceptable salt of any of the foregoing.
   (15) The compound of embodiment (1), selected from:

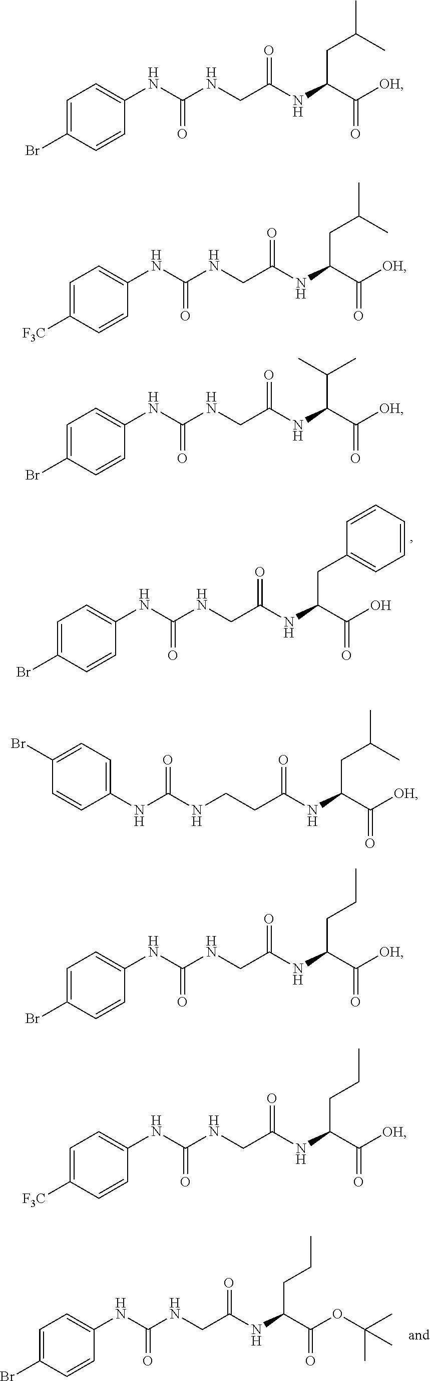

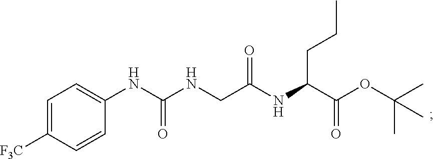 [SMILES: CCC[C@H](NC(=O)CNC(=O)Nc1ccc(C(F)(F)F)cc1)C(=O)OC(C)(C)C]

and tautomers thereof;
and pharmaceutically acceptable salts thereof;
preferably, the compounds are selected from:

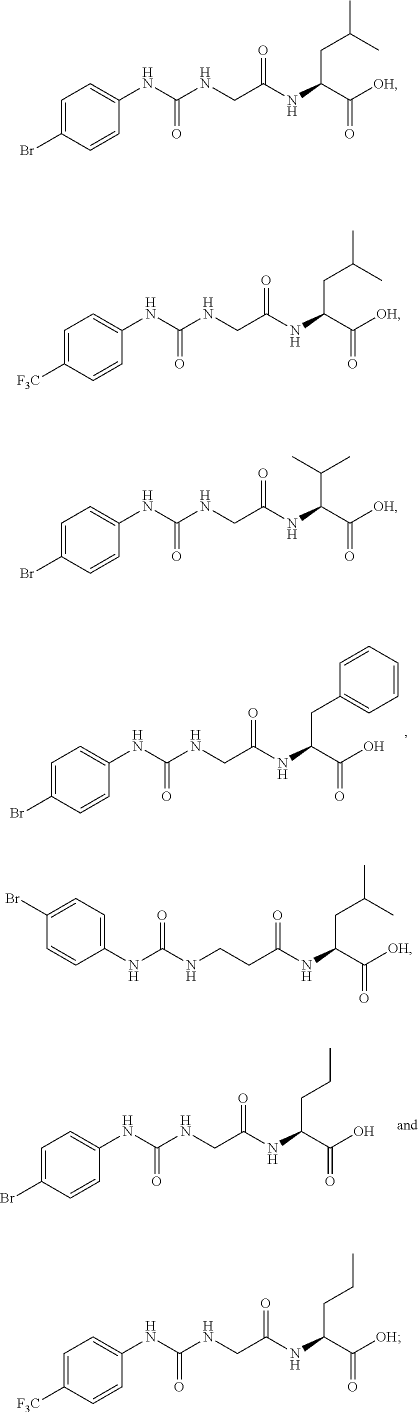

and pharmaceutically acceptable salts thereof.
(16) A pharmaceutical composition comprising a compound of any one of embodiments (1) through (15) and a pharmaceutically acceptable excipient.
(17) A method of treating a disease or condition alleviated by FPR modulation, the method comprising administering a therapeutically effective amount of a compound or composition of any one of embodiments (1) through (16) to a subject in need of such treatment.
(18) The method of embodiment (17), wherein the disease or condition is an ocular disease or condition selected from: ocular inflammation, age-related macular degeneration, wet macular degeneration, dry macular degeneration, uveitis, dry eye, keratitis, allergic eye disease and conditions affecting the posterior part of the eye, such as maculopathies and retinal degeneration including non-exudative age related macular degeneration, exudative age related macular degeneration, choroidal neovascularization, diabetic retinopathy (proliferative), retinopathy of prematurity, acute macular neuroretinopathy, central serous chorioretinopathy, cystoid macular edema, and diabetic macular edema; infectious keratitis, herpetic keratitis, corneal angiogenesis, lymphangiogenesis, retinitis, and choroiditis such as acute multifocal placoid pigment epitheliopathy, Behcet's disease, birdshot retinochoroidopathy, infectious (syphilis, lyme, tuberculosis, toxoplasmosis), intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi- and Harada syndrome; vascular diseases/exudative diseases such as retinal arterial occlusive disease, central retinal vein occlusion, cystoids macular edema, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angiitis, sickle cell retinopathy and other hemoglobinopathies, angioid streaks, familial exudative vitreoretinopathy, and Eales disease; traumatic/surgical conditions such as sympathetic ophthalmia, uveitic retinal disease, retinal detachment, trauma, conditions caused by laser, conditions caused by photodynamic therapy, photocoagulation, hypoperfusion during surgery, radiation retinopathy, bone marrow transplant retinopathy, post-surgical corneal wound healing or inflammation, and post-cataract surgical inflammation; proliferative disorders such as proliferative vitreal retinopathy and epiretinal membranes, and proliferative diabetic retinopathy; infectious disorders such as ocular histoplasmosis, ocular toxocariasis, presumed ocular histoplasmosis syndrome (POHS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; and miscellaneous other diseases affecting the posterior part of the eye such as punctate inner choroidopathy, acute posterior multifocal placoid pigment epitheliopathy, myopic retinal degeneration, and acute retinal pigment epitheliitis; post-surgical corneal inflammation, blepharitis, meibomian gland dysfunction (MGD), corneal wound healing, glaucoma, branch vein occlusion, Best's vitelliform macular degeneration, retinitis pigmentosa, proliferative vitreoretinopathy (PVR), and any other degenerative disease of either the photoreceptors or the retinal pigment epithelium; preferably, the ocular disease or condition is selected from: dry eye, a post-surgical corneal wound, post-surgical corneal inflammation, and post-cataract surgical inflammation.
(19) The method of embodiment (17), wherein the disease or condition is a dermal disease or condition selected from: dermal inflammation, a dermal wound, hypertrophic scars, keloids, burns, sunburn, rosacea, erythema of the skin, atopic dermatitis, acne, psoriasis, seborrheic dermatitis, actinic keratoses, basal cell carcinoma, squamous cell carcinoma, melanoma, viral warts, photoaging, photodamage, melasma, post-inflammatory hyperpigmentation, disorders of pigmentation, alopecia, scarring and non-scarring forms; preferably, the dermal disease or condition is psoriasis or rosacea.
(20) The method of embodiment (17), wherein the disease or condition is selected from a systemic inflammatory disease or condition, stroke, coronary artery disease, a cardiovascular disorder, coronary artery disease and angina pectoris.
(21) The method of embodiment (17), wherein the disease or condition is an obstructive airway disease.
(22) The method of embodiment (17), wherein the disease or condition is a neurological disorder, a central nervous system disorder, Alzheimer's disease, neuroinflammation or pain.
(23) The method of embodiment (17), wherein the disease or condition is an HIV-mediated retroviral infection.
(24) The method of embodiment (17), wherein the disease or condition is an immunological disorder, arthritis, rheumatoid arthritis, systemic lupus erythematosus, or multiple sclerosis.
(25) The method of embodiment (17), wherein the disease or condition is sepsis.
(26) The method of embodiment (17), wherein the disease or condition is inflammatory bowel disease or ulcerative colitis.
(27) The method of embodiment (17), wherein the disease or condition is asthma or an allergic disorder.
(28) The method of embodiment (17), wherein the disease or condition is cachexia.
(29) The method of any one of embodiments (17) through (28), wherein the subject is a human.
(30) A method of selectively modulating an FPR1 receptor relative to an FPR2 receptor in a recipient, the method comprising administering at least one compound of the invention (a compound of Formula I, Ia, II or IIa) to the recipient, wherein the compound exhibits at least 2-fold selectivity for FPR1 relative to FPR2, and wherein the selectivity is based on the ratio of the EC₅₀ for agonizing FPR2 to the EC₅₀ for agonizing FPR1 as measured in an in vitro, ex vitro and/or in vivo assay.
(31) The method of embodiment (30), wherein the recipient is a mammalian subject.
(32) The method of embodiment (31), wherein the subject is a human.
(33) The method of embodiment (30), wherein the recipient is a cell or tissue.
(34) The method of embodiment (33), wherein the administration is to a cell or tissue in vitro or ex vivo.
(35) The method of any one of embodiments (30) through (34), wherein the at least one compound exhibits at least 5-fold selectivity for FPR1 compared to FPR2.
(36) The method of any one of embodiments (30) through (34), wherein the at least one compound exhibits at least 10-fold selectivity for FPR1 compared to FPR2.
(37) The method of any one of embodiments (30) through (34), wherein the at least one compound exhibits at least 20-fold selectivity for FPR1 compared to FPR2.
(38) The method of any one of embodiments (30) through (34), wherein the at least one compound exhibits at least 50-fold selectivity for FPR1 compared to FPR2.
(39) The method of any one of embodiments (30) through (34), wherein the at least one compound exhibits at least 100-fold selectivity for FPR1 compared to FPR2.
(40) The method of any one of embodiments (30) through (34), wherein the at least one compound exhibits at least 200-fold selectivity for FPR1 compared to FPR2.

The present invention concerns also processes for preparing the compounds of Formula I, Ia, II and IIa. Synthetic Scheme 1a, set forth below, illustrates how the compounds according to the invention can be made. Further examples are provided in Scheme 1b.

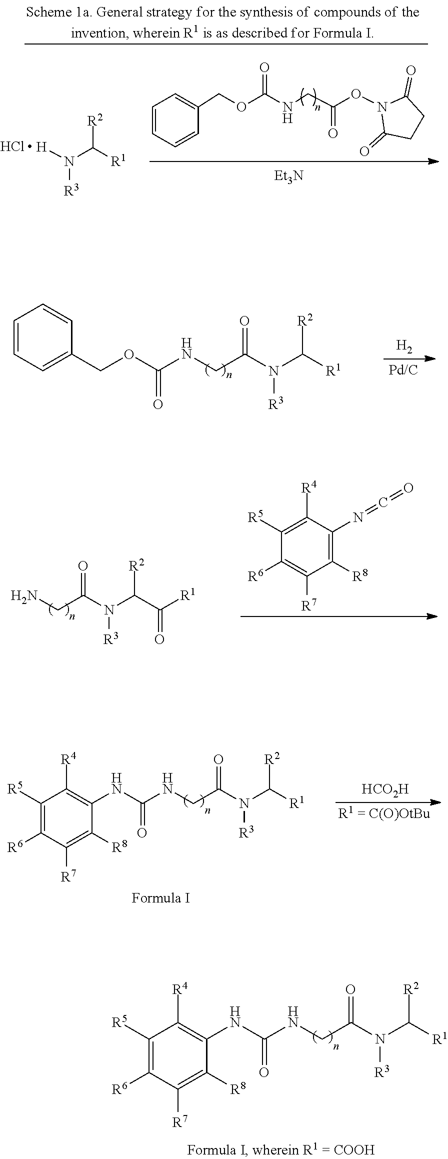

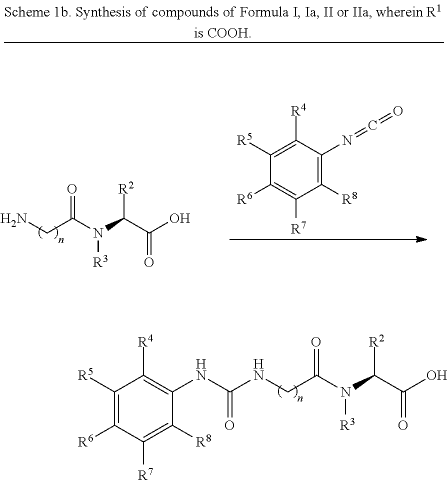

Those skilled in the art will be able to routinely modify and/or adapt Scheme 1a and/or 1b to synthesize any compounds of the invention that fall within the scope of Formula I, Ia, II or IIa.

Each and every feature described herein, and each and every combination of two or more of such features, is included within the scope of the present invention provided that the features included in such a combination are not mutually inconsistent.

The following Examples illustrate how compounds according to the invention can be made, and provide details of certain specific chemical transformations. The Examples are for illustrative purposes only and are not intended, nor should they be construed, as limiting the invention in any manner. Those skilled in the art will be able to routinely modify and/or adapt the Examples to synthesize any compound of the invention covered by Formula I, Ia, II and IIa, and will appreciate that variations and modifications of the Examples can be made without exceeding the spirit or scope of the invention.

The following abbreviations are used herein:

CD₃OD deuterated methanol
Et₃N triethylamine
EtOAc ethyl acetate
H₂ hydrogen gas
HCO₂H formic acid
Na₂SO₄ sodium sulfate
Pd/C palladium on carbon
THF tertahydrofuran
TMS tetramethylsilane

All reagents, solvents and catalysts for which the synthesis is not described are purchased from chemical vendors such as 3B Scientific, Sigma Aldrich, Fluka, Bio-Blocks, Combi-blocks, TCI, VWR, Lancaster, Oakwood, Trans World Chemical, Alfa, Fisher, Maybridge, Frontier, Matrix, Ukrorgsynth, Toronto, Ryan Scientific, SiliCycle, Anaspec, Syn Chem, Chem-Impex, MIC-scientific, Ltd; however some known intermediates were prepared according to published procedures.

Compound names were generated with ACDLab version 12.5; some intermediate and reagent names used in the Examples were generated with software such as Chem Bio Draw Ultra version 12.0, ACDLab version 12.5 or Auto Nom 2000 from MDL ISIS Draw 2.5 SP1.

In general, characterization of the compounds was performed using NMR spectroscopy. NMR spectra were acquired on a 300 or 600 MHz Varian NMR spectrometer and at room temperature. Chemical shifts are given in ppm referenced either to internal TMS or to the solvent signal.

Usually, the compounds of the invention were purified by medium pressure liquid chromatography, unless noted otherwise.

### Example A

### Intermediate 1

### (S)-tert-butyl 2-(2(((benzyloxy)carbonyl)amino)acetamido)pentanoate

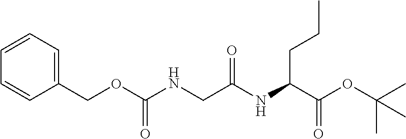 [SMILES: CCC[C@H](NC(=O)CNC(=O)OCc1ccccc1)C(=O)OC(C)(C)C]

To a solution of L-norvaline (500 mg, 2.39 mmol) and 12 mL of THF at 25° C. was added 2,5-dioxopyrrolidin-1-yl-2-((benzyloxy)carbonyl)amino acetate (730 mg, 2.39 mmol) and triethylamine (0.66 mL, 4.78 mmol). The resulting mixture was stirred at 25° C. for 2 hours. The mixture was quenched with water (5 mL) then extracted with ethyl acetate (30 mL). The layers were separated, and the organic layer was washed with water, brine, dried over Na₂SO₄, filtered, and concentrated under reduced pressure. The resulting product was purified by medium pressure liquid chromatography on silica gel using ethyl acetate:hexanes (1:1) to yield Intermediate 1 as clear oil. ¹H NMR (CD₃OD, 600 MHz) δ: 7.31-7.37 (m, 4H), 7.26-7.29 (m, 1H), 5.09 (s, 2H), 4.24-4.33 (m, 1H), 3.74-3.87 (m, 2H), 1.69-1.79 (m, 1H), 1.59-1.67 (m, 1H), 1.44 (s, 9H), 1.32-1.41 (m, 2H), 0.92 (t, J=7.3 Hz, 3H).

### Example B

### Intermediate 2

### (S)-tert-butyl 2-(2-aminoacetamido)pentanoate

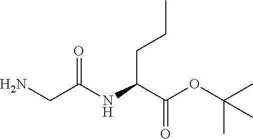 [SMILES: CCC[C@H](NC(=O)CN)C(=O)OC(C)(C)C]

To a solution of Intermediate 1 (760 mg, 2.09 mmol) and 35 mL of methanol at 25° C. was added 10% palladium on carbon (85 mg) and a hydrogen balloon. The resulting mixture was stirred at 25° C. for 12 hours. The mixture was filtered through Celite® and concentrated under reduced pressure to yield Intermediate 2 as a clear oil. ¹H NMR (CD₃OD, 600 MHz) b: 4.23-4.34 (m, 1H), 3.29-3.30 (m, 2H), 1.70-1.80 (m, 1H), 1.59-1.68 (m, 1H), 1.45 (s, 9H), 1.35-1.43 (m, 2H), 0.91-0.97 (m, 3H).

### Example 1

### Compound 1

### (S)-2-(2-(3-(4-bromophenyl)ureido)acetamido)-4-methylpentanoic acid

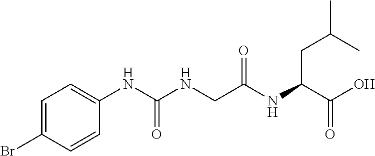 [SMILES: CC(C)C[C@H](NC(=O)CNC(=O)Nc1ccc(Br)cc1)C(=O)O]

To a solution of glycyl-L-leucine (150 mg, 0.80 mmol) and 12 mL of methylene chloride at 25° C. was added 4-bromo-phenyl isocyanate (157 mg, 0.80 mmol) and triethylamine (0.17 mL, 1.20 mmol). The resulting mixture was stirred at 25° C. for 12 hours. The mixture was concentrated and the residue was purified by medium pressure liquid chromatography on silica gel using methanol: dichloromethane (15:85) to yield Compound 1 as white solid. ¹H NMR (CD₃OD, 300 MHz) b: 7.25-7.41 (m, 4H), 4.41-4.55 (m, 1H), 3.81-3.99 (m, 2H), 1.59-1.83 (m, 3H), 0.89-1.02 (m, 6H).

Compounds 2, 3, 4 and 5 were prepared from the corresponding amino acid in a similar manner to the procedure described for Example 1. Intermediate (H-beta-Ala-Leu-OH), purchased from 3B Scientific, was used to prepare Compound 5.

### Example 2

### Compound 2

### (S)-4-methyl-2-(2-(3-(4-(trifluoromethyl)phenyl)ureido)acetamido)pentanoic acid

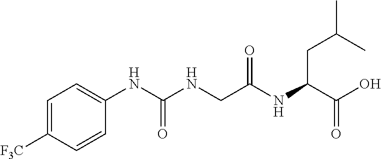 [SMILES: CC(C)C[C@H](NC(=O)CNC(=O)Nc1ccc(C(F)(F)F)cc1)C(=O)O]

¹H NMR (CD₃OD, 600 MHz) δ: 7.45-7.62 (m, 4H), 4.46 (dd, J=9.4, 5.3 Hz, 1H), 3.86-4.00 (m, 2H), 1.61-1.80 (m, 3H), 0.94 (dd, J=11.2, 6.5 Hz, 6H).

### Example 3

### Compound 3

### (S)-2-(2-(3-(4-bromophenyl)ureido)acetamido)-3-methylbutanoic acid

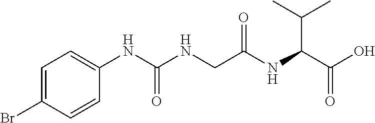 [SMILES: CC(C)[C@H](NC(=O)CNC(=O)Nc1ccc(Br)cc1)C(=O)O]

¹H NMR (CD₃OD, 600 MHz) δ: 7.35-7.39 (m, 2H), 7.30-7.34 (m, 2H), 4.36 (d, J=5.3 Hz, 1H), 3.87-3.97 (m, 2H), 2.16-2.23 (m, 1H), 0.98 (d, J=7.0 Hz, 3H), 0.96 (d, J=7.0 Hz, 3H).

### Example 4

### Compound 4

### (S)-2-(2-(3-(4-bromophenyl)ureido)acetamido)-3-phenylpropanoic acid

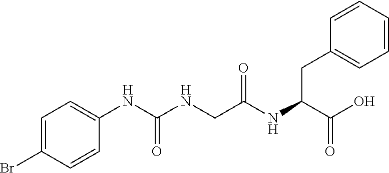 [SMILES: O=C(CNC(=O)Nc1ccc(Br)cc1)N[C@@H](Cc1ccccc1)C(=O)O]

¹H NMR (CD₃OD, 600 MHz) δ: 7.37 (d, J=8.8 Hz, 2H), 7.30-7.33 (m, 2H), 7.16-7.22 (m, 5H), 4.67 (dd, J=7.6, 5.3 Hz, 1H), 3.85-3.92 (m, 1H), 3.76-3.81 (m, 1H), 3.15-3.20 (m, 1H), 3.03 (dd, J=14.1, 7.6 Hz, 1H).

### Example 5

### Compound 5

### (S)-2-(3-(3-(4-bromophenyl)ureido)propanamido)-4-methylpentanoic acid

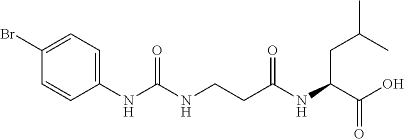 [SMILES: CC(C)C[C@H](NC(=O)CCNC(=O)Nc1ccc(Br)cc1)C(=O)O]

¹H NMR (CD₃OD, 600 MHz) δ: 7.33-7.36 (m, 2H), 7.27-7.30 (m, 2H), 4.42 (t, J=6.7 Hz, 1H), 3.39-3.51 (m, 2H), 2.41-2.55 (m, 2H), 1.64-1.73 (m, 1H), 1.58-1.63 (m, 2H), 0.91 (dd, J=12.3, 6.5 Hz, 6H).

### Example 6

### Compound 6

### (S)-2-(2-(3-(4-bromophenyl)ureido)acetamido)pentanoic acid

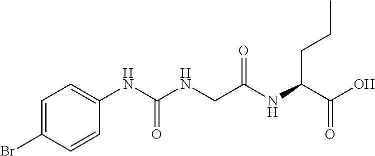 [SMILES: CCC[C@H](NC(=O)CNC(=O)Nc1ccc(Br)cc1)C(=O)O]

A solution of Compound 8 (220 mg, 0.51 mmol) and 8 mL of formic acid was stirred at 25° C. for 12 hours. The resulting reaction was quenched with water (10 mL), and the product was extracted with EtOAc. The organic layer was washed with water, brine, dried over Na₂SO₄, filtered, and concentrated under reduced pressure. The residue was rinsed two times with acetone: hexane (2:98) to yield Compound 6 as a white solid. ¹H NMR (CD₃OD, 600 MHz) δ: 7.35 (s, 2H), 7.30 (d, J=9.4 Hz, 2H), 4.38-4.45 (m, 1H), 3.83-3.97 (m, 2H), 1.79-1.87 (m, 1H), 1.64-1.73 (m, 1H), 1.36-1.47 (m, 2H), 0.90-0.98 (m, 3H).

### Example 7

### Compound 7

### (S)-2-(2-(3-(4-(trifluoromethyl)phenyl)ureido)acetamido)pentanoic acid

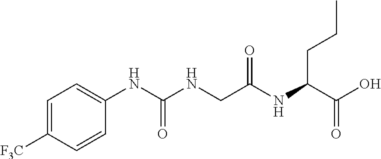 [SMILES: CCC[C@H](NC(=O)CNC(=O)Nc1ccc(C(F)(F)F)cc1)C(=O)O]

Compound 7 was prepared from the corresponding tert-butyl ester in a similar manner to the procedure described for Compound 6. ¹H NMR (CD₃OD, 600 MHz) δ: 7.54-7.57 (m, 2H), 7.50-7.53 (m, 2H), 4.37-4.44 (m, 1H), 3.88-3.98 (m, 2H), 1.82 (dddd, J=13.9, 9.3, 6.7, 4.7 Hz, 1H), 1.64-1.74 (m, 1H), 1.35-1.48 (m, 2H), 0.93 (t, J=7.3 Hz, 3H).

### Example 8

### Compound 8

### (S)-tert-butyl 2-(2-(3-(4-bromophenyl)ureido)acetamido)pentanoate

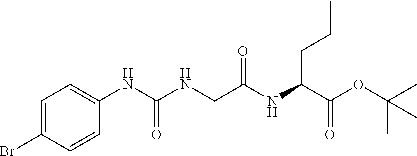 [SMILES: CCC[C@H](NC(=O)CNC(=O)Nc1ccc(Br)cc1)C(=O)OC(C)(C)C]

To a solution of Intermediate 2 (182 mg, 0.79 mmol) and 7 mL of methylene chloride at 25° C. was added 4-bromo-phenyl isocyanate (156 mg, 0.79 mmol) and triethylamine (0.17 mL, 1.60 mmol). The resulting mixture was stirred at 25° C. for 12 hours. The mixture was concentrated and the residue was purified by medium pressure liquid chromatography on silica gel using ethyl acetate:hexane (1:1) to yield Compound 8 as white solid. ¹H NMR (CD₃OD, 600 MHz) b: 7.35 (s, 2H), 7.29-7.32 (m, 2H), 4.24-4.33 (m, 1H), 3.89 (q, J=16.6 Hz, 2H), 1.72-1.81 (m, 1H), 1.60-1.69 (m, 1H), 1.44 (s, 9H), 1.34-1.41 (m, 2H), 0.93 (t, J=7.3 Hz, 3H).

### Example 9

### Compound 9

### (S)-tert-butyl 2-(2-(3-(4-(trifluoromethyl)phenyl)ureido)acetamido) pentanoate

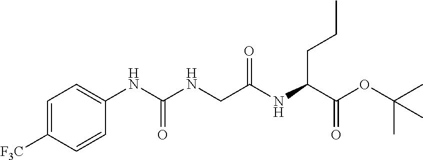 [SMILES: CCC[C@H](NC(=O)CNC(=O)Nc1ccc(C(F)(F)F)cc1)C(=O)OC(C)(C)C]

Compound 9 was prepared from the corresponding amine in a similar manner to the procedure described for Compound 8. ¹H NMR (CD₃OD, 600 MHz) b: 7.54-7.57 (m, 2H), 7.50-7.52 (m, 2H), 4.26-4.32 (m, 1H), 3.86-3.98 (m, 2H), 1.73-1.80 (m, 1H), 1.61-1.69 (m, 1H), 1.45 (s, 9H), 1.37-1.43 (m, 2H), 0.93 (t, J=7.3 Hz, 3H).

### Biological Data

Biological activity of some specific compounds of the invention is set forth in Table 1 below. CHO-Gα16 cells stably expressing FPR1 or FPR2 were cultured in (F12, 10% FBS, 1% PSA, 400 μg/ml geneticin and 50 μg/ml hygromycin). In general, the day before the experiment, 18,000 cells/well were plated in a 384-well clear bottom poly-D-lysine coated plate. The following day the screening compound-induced calcium activity was assayed on the FLIPR^{Tetra}. The drug plates were prepared in 384-well microplates using the EP3 and the MultiPROBE robotic liquid handling systems. Compounds were tested at concentrations ranging from 0.61 to 10,000 nM. Results are expressed as EC₅₀ (nM) and efficacy values.

TABLE 1

| | | FPR2 | FPR1 |
|---|---|---|---|
| | | Gal6- | Gal6- |
| | | HEK293 | HEK293 |
| | IUPAC Name | EC₅₀ (nM) | EC₅₀ (nM) |
| Structure | Compound | (Rel. eff.) | (Rel. eff.) |
| 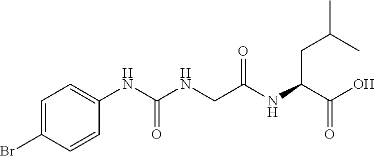 [SMILES: CC(C)C[C@H](NC(=O)CNC(=O)Nc1ccc(Br)cc1)C(=O)O] | (S)-2-(2-(3-(4- bromophenyl)ureido) acetamido)-4-methyl- pentanoic acid | 224 nM (0.91) | 0.58 nM (1.00) |
| 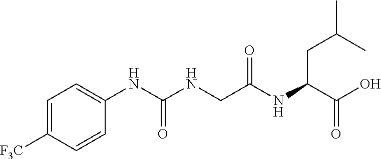 [SMILES: CC(C)C[C@H](NC(=O)CNC(=O)Nc1ccc(C(F)(F)F)cc1)C(=O)O] | (S)-4-methy1-2-(2-(3-(4- (trifluoromethyl)phenyl) ureido)acetamido)pentanoic acid | 213 nM (0.96) | 0.59 nM (0.96) |
| 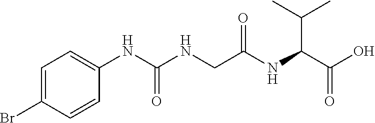 [SMILES: CC(C)[C@H](NC(=O)CNC(=O)Nc1ccc(Br)cc1)C(=O)O] | (S)-2-(2-(3-(4- bromophenyl)ureido) acetamido)-3-methylbutanoic acid | 241 nM (0.96) | 1 nM (1.04) |
| 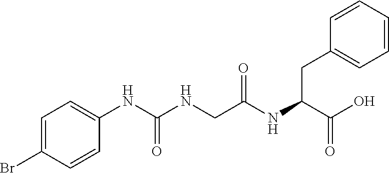 [SMILES: O=C(CNC(=O)Nc1ccc(Br)cc1)N[C@@H](Cc1ccccc1)C(=O)O] | (S)-2-(2-(3-(4- bromophenyl)ureido) acetamido)-3-phenyl- propanoic acid | 356 nM (1.07) | 14.7 nM (0.97) |
| 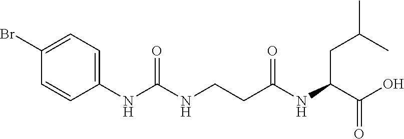 [SMILES: CC(C)C[C@H](NC(=O)CCNC(=O)Nc1ccc(Br)cc1)C(=O)O] | (S)-2-(3-(3-(4- bromophenyl)ureido) propanamido)-4- methylpentanoic acid | 5629 nM (0.81) | 76 nM (0.99) |
| 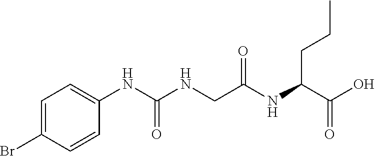 [SMILES: CCC[C@H](NC(=O)CNC(=O)Nc1ccc(Br)cc1)C(=O)O] | (S)-2-(2-(3-(4- bromophenyl)ureido) acetamido)pentanoic acid | 308 nM (1.05) | 4.93 nM (0.98) |
| 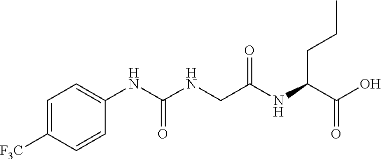 [SMILES: CCC[C@H](NC(=O)CNC(=O)Nc1ccc(C(F)(F)F)cc1)C(=O)O] | (S)-2-(2-(3-(4- (trifluoromethyl)phenyl) ureido)acetamido)pentanoic acid | 554 nM (1.01) | 3.29 nM (0.90) |
| 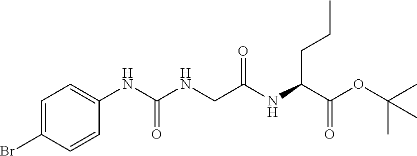 [SMILES: CCC[C@H](NC(=O)CNC(=O)Nc1ccc(Br)cc1)C(=O)OC(C)(C)C] | (S)-tert-butyl 2-(2-(3-(4- bromophenyl)ureido) acetamido)pentanoate | 598 nM (0.97) | 2303 nM (0.82) |
| 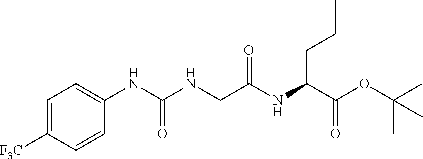 [SMILES: CCC[C@H](NC(=O)CNC(=O)Nc1ccc(C(F)(F)F)cc1)C(=O)OC(C)(C)C] | (S)-tert-butyl 2-(2-(3-(4- (trifluoromethyl)phenyl) ureido)acetamido)pentanoate | 1248 nM (1.00) | 5145 nM (0.92) |

1. A compound of Formula I: 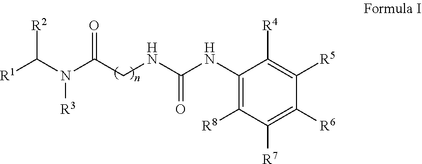
wherein:
R¹ is —COOH, —C(O)OR^{a}, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole; and R^{a} is optionally substituted C₁₋₆ alkyl; wherein said optional alkyl substituent is selected from OH, halogen, —OC₁₋₈alkyl and —(O(CH₂)₁₋₈)_{q}—OC₁₋₈ alkyl; and q is 1, 2, 3, 4, 5 or 6;
R² is optionally substituted C₁₋₆alkyl, wherein said optional alkyl substituent is selected from —OH, —SH, —OC₁₋₆ alkyl, —SC₁₋₆ alkyl, —COOH, —C(O)OC₁₋₆ alkyl, —C(O)NH₂, optionally substituted C₃₋₈cycloalkyl, optionally substituted C₃₋₈cycloalkenyl, optionally substituted C₆₋₁₀aryl, and optionally substituted heterocycle;
R³ is H or unsubstituted C₁₋₆ alkyl;
R⁴ is H, optionally substituted C₁₋₆ alkyl, C₁₋₆ haloalkyl, optionally substituted C₃₋₈ cycloalkyl, optionally substituted C₃₋₈ cycloalkenyl, optionally substituted C₆₋₁₀ aryl, optionally substituted heterocycle, halogen, —NR₁₁R¹², —S(O)ₘR⁹, —C(O)R¹⁰, —C(O)OC₁₋₆ alkyl, —C(O)SC₁₋₆ alkyl, —OR¹³ or —SR¹³;
R⁵ is H, optionally substituted C₁₋₆alkyl, C₁₋₆haloalkyl, halogen, —S(O)ₘR⁹ or —C(O)R¹⁰;
R⁶ is optionally substituted C₁₋₆alkyl, C₁₋₆haloalkyl, optionally substituted C₃₋₈ cycloalkyl, optionally substituted C₃₋₈cycloalkenyl, optionally substituted C₆₋₁₀ aryl, optionally substituted heterocycle, halogen, —S(O)ₘR⁹, —C(O)R¹⁰ or —OR¹¹;
R⁷ is H, optionally substituted C₁₋₆alkyl, C₁₋₆haloalkyl, halogen, —S(O)ₘR⁹ or —C(O)R¹⁰;
R⁸ is H, optionally substituted C₁₋₆ alkyl, C₁₋₆ haloalkyl, optionally substituted C₃₋₈ cycloalkyl, optionally substituted C₃₋₈ cycloalkenyl, optionally substituted C₆₋₁₀ aryl, optionally substituted heterocycle, halogen, —NR₁₁R¹², —S(O)ₘR⁹, —C(O)R¹⁰, —C(O)OC₁₋₆ alkyl, —C(O)SC₁₋₆ alkyl, —OR¹³ or —SR¹³;
each R⁹ is independently —OH, optionally substituted C₁₋₆alkyl or optionally substituted C₆₋₁₀aryl;
each R¹⁰ is independently —OH, optionally substituted C₁₋₆alkyl or optionally substituted C₆₋₁₀aryl;
each R¹¹ is independently H, optionally substituted C₁₋₈alkyl, optionally substituted C₃₋₈ cycloalkyl, optionally substituted C₃₋₈cycloalkenyl, optionally substituted C₆₋₁₀ aryl or optionally substituted heterocycle;
each R¹² is independently H, optionally substituted C₁₋₈alkyl, optionally substituted C₃₋₈ cycloalkyl, optionally substituted C₃₋₈cycloalkenyl, optionally substituted C₆₋₁₀aryl or optionally substituted heterocycle;
each R¹³ is independently H or optionally substituted C₁-8 alkyl;
each m is independently 1 or 2; and
n is 1, 2 or 3;
or a single enantiomer thereof;
or a mixture of enantiomers thereof;
or a tautomer of the foregoing;
or pharmaceutically acceptable salt of the foregoing;
provided that the compound is not:

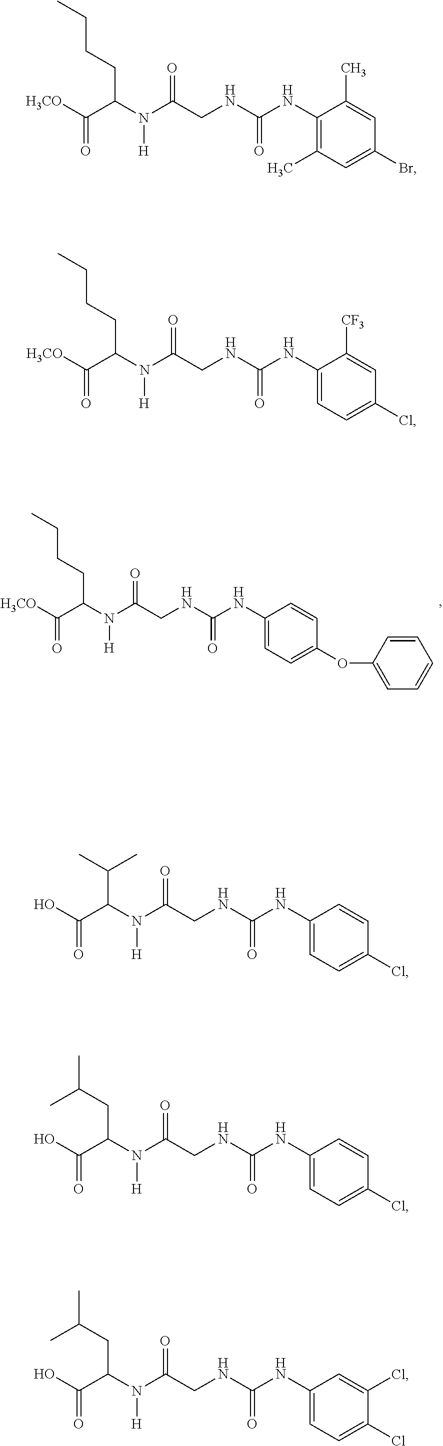 [SMILES: CCCCC(NC(=O)CNC(=O)Nc1c(C)cc(Br)cc1C)C(=O)OC ; CCCCC(NC(=O)CNC(=O)Nc1ccc(Cl)cc1C(F)(F)F)C(=O)OC ; CCCCC(NC(=O)CNC(=O)Nc1ccc(Oc2ccccc2)cc1)C(=O)OC ; CC(C)C(NC(=O)CNC(=O)Nc1ccc(Cl)cc1)C(=O)O ; CC(C)CC(NC(=O)CNC(=O)Nc1ccc(Cl)cc1)C(=O)O ; CC(C)CC(NC(=O)CNC(=O)Nc1ccc(Cl)c(Cl)c1)C(=O)O]

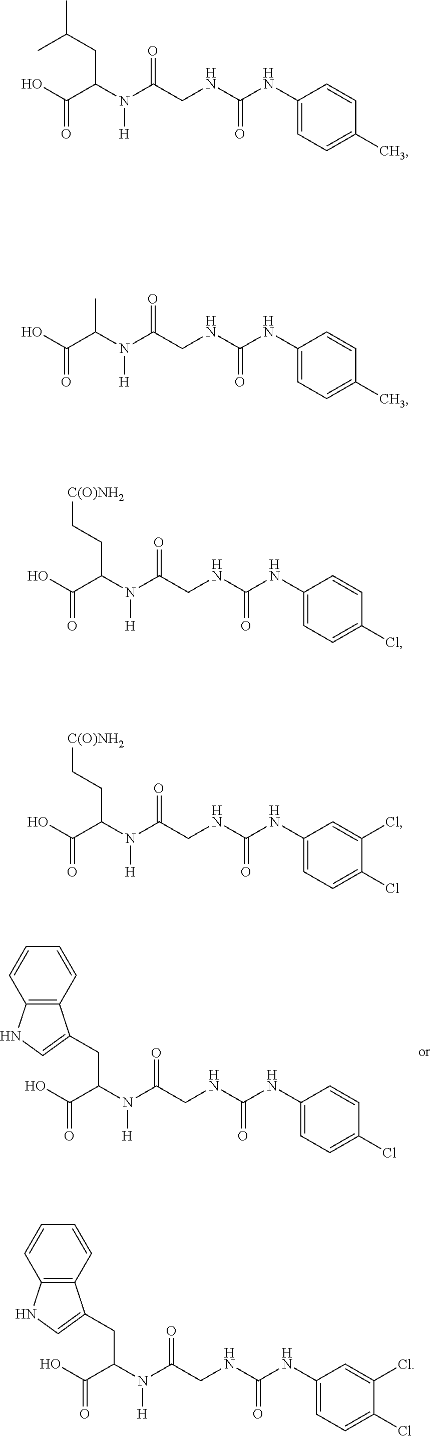

2. The compound of claim 1, wherein R¹ is —COOH, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole.

3. The compound of claim 1, wherein R¹ is —COOH.

4. The compound of claim 1, wherein R² is unsubstituted C₁₋₆alkyl or benzyl.

5. The compound of claim 1, wherein R³ is H or methyl.

6. The compound of claim 1, wherein n is 1 or 2.

7. The compound of claim 1, wherein R⁴ is H, F or C₁₋₆haloalkyl; R⁵ is H, F or C₁₋₆haloalkyl; R⁷ is H, F or C₁₋₆haloalkyl; and R⁸ is H, F or C₁₋₆ haloalkyl.

8. The compound of claim 1, wherein each of R⁴, R⁵, R⁷ and R⁸ is H.

9. The compound of claim 1, wherein R⁶ is C₁₋₆haloalkyl or halogen.

10. The compound of claim 1, provided that R⁶ is not chlorine, methyl or —OC₆₋₁₀aryl.

11. The compound of claim 1, wherein:
R¹ is —COOH, —C(O)OR^{a}, sulfonate, sulfonic acid, phosphonate, phosphonic acid, phosphoric acid, boronic acid or an optionally substituted heterocycle, wherein said heterocycle is selected from tetrazole, imidazole, thiazole, oxazole, triazole, isoxazole, oxadiazole, thiadiazole, thiophene, pyrazole and pyrrole;
wherein R^{a} is optionally substituted C₁₋₆ alkyl, wherein said optional alkyl substituent is selected from OH, halogen, —OC₁₋₈ alkyl and —(O(CH₂)₁₋₈)_{q}—OC₁₋₈ alkyl; and q is 1, 2, 3, 4, 5 or 6;
R² is unsubstituted C₁₋₆ alkyl or benzyl;
R³ is H or unsubstituted C₁₋₆ alkyl;
R⁴ is H or halogen;
R⁵ is H;
R⁶ is C₁₋₆ haloalkyl or bromine;
R⁷ is H;
R⁸ is H or halogen; and
n is 1 or 2;
or a single enantiomer thereof;
or a mixture of enantiomers thereof;
or a tautomer of the foregoing;
or pharmaceutically acceptable salt of the foregoing.

12. The compound of claim 11, wherein R¹ is —COOH.

13. The compound of claim 11, wherein R⁴ and R⁸ is H.

14. The compound of claim 11, wherein:
R¹ is —COOH or —C(O)OR^{a}, wherein R^{a} is unsubstituted C₁₋₆ alkyl;
R² is unsubstituted C₁₋₃ alkyl or benzyl;
R³ is H or —CH₃;
R⁴ is H;
R⁵ is H;
R⁶ is —CF₃ or bromine;
R⁷ is H;
R⁸ is H; and
n is 1 or 2;
or a tautomer thereof;
or pharmaceutically acceptable salt of any of the foregoing.

15. The compound of claim 1, selected from the group consisting of: 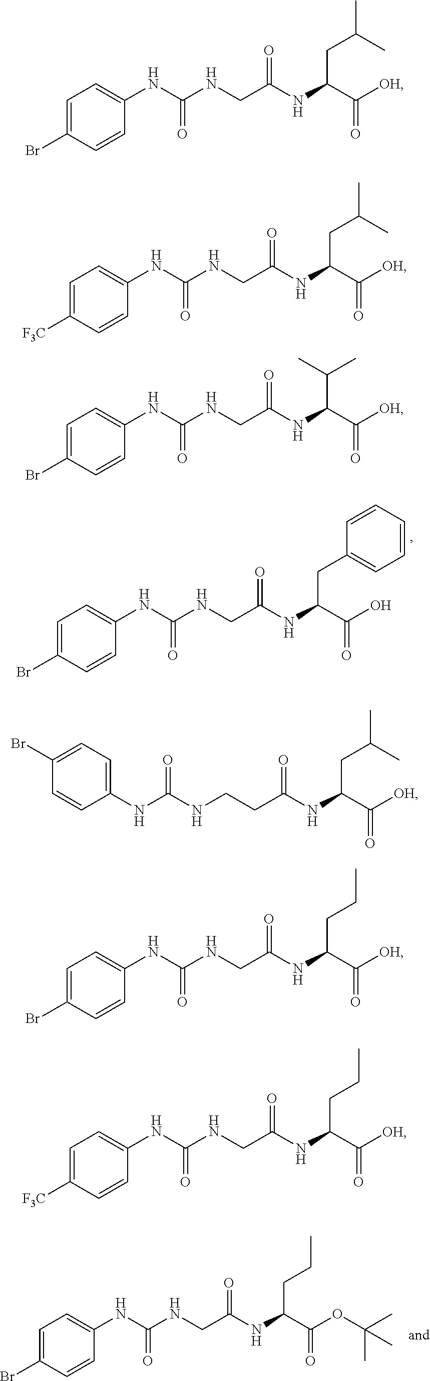 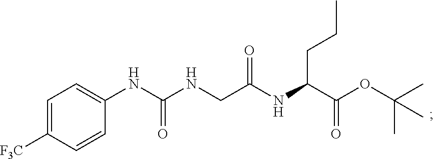 [SMILES: CCC[C@H](NC(=O)CNC(=O)Nc1ccc(C(F)(F)F)cc1)C(=O)OC(C)(C)C]
and tautomers thereof;
and pharmaceutically acceptable salts thereof.

16. A pharmaceutical composition comprising a compound of claim 1 and a pharmaceutically acceptable excipient.

17.-20. (canceled)
